(19) United States
(12) Patent Application Publication
(10) Pub. No.: US 2006/0141504 A1
(43) Pub. Date: Jun. 29, 2006

(54) Molecular technologies for improved risk classification and therapy for acute lymphoblastic leukemia in children and adults

(76) Inventors: Cheryl L. Willman, Albuquerque, NM (US); Edward Bedrick, Albuquerque, NM (US); Huining Kang, Albuquerque, NM (US); Paul Helman, Albuquerque, NM (US); Robert Veroff, Albuquerque, NM (US)

Correspondence Address: COLEMAN SUDOL SAPONE, P.C., 714 COLORADO AVENUE, BRIDGE PORT, CT, 06605-1601, US

(21) Appl. No.: 11/285,976

(22) Filed: Nov. 23, 2005

Related U.S. Application Data
(60) Provisional application No. 60/630,298, filed on Nov. 23, 2004.
(60) Provisional application No. 60/720,410, filed on Sep. 26, 2005.

(51) Int. Cl. C12Q 1/68

(52) U.S. Cl. 435/6

(57) ABSTRACT

The present invention relates to methods for predicting the outcome of therapeutic intervention in cases of leukemia, especially acute lymphoblastic leukemia in children and adults. The present invention evaluates a gene expression profile and identifies prognostic genes of cancers, in particular leukemia, more particularly B-precursor acute lymphoblastic leukemia (ALL). The present invention provides a method of determining prognosis of leukemia, in particular, acute lymphoblastic leukemia, more particularly B-precursor ALL and predicting therapeutic outcome of a patient. The method comprises the steps of first establishing the threshold value of at least three prognostic genes of leukemia, preferably at least eight prognostic genes, or preferably, as many as 26 prognostic genes. Then, the amount of the prognostic gene(s) from a leukemia patient is determined. The amount of the prognostic gene present in that patient is compared with the established threshold value of the prognostic gene(s) which is indicative of therapeutic success or failure, whereby the prognostic outcome of the patient is determined/predicted.

### RELATED APPLICATIONS AND SUPPORT

This application claims the benefit of priority of provisional applications U.S. Ser. No. 60/630,298, filed Nov. 23, 2004 and U.S. Ser. No. 60/720,410, filed Sep. 26, 2005, both of which applications are incorporated by reference in their entirety herein.

The invention described in this application was made with support from the National Institutes of Health (National Cancer Institute), Grant No. NIH NCI U01 CA88461; and under a contract from the Department of Energy, Contract No. DE-AC04-94AL85000. The U.S. Government retains certain rights in this invention.

### FIELD OF THE INVENTION

The present invention relates to methods for predicting the outcome of therapeutic intervention in cases of leukemia, especially acute lymphoblastic leukemia in children and adults.

### BACKGROUND AND DESCRIPTION LEADING UP TO THE INVENTION

Leukemia is the most common childhood malignancy in the United States. Approximately 3,500 cases of acute leukemia are diagnosed each year in the U.S. in children less than 20 years of age. The large majority (>70%) of these cases are acute lymphoblastic leukemias (ALL) and the remainder acute myeloid leukemias (AML). The outcome for children with ALL has improved dramatically over the past three decades, but despite significant progress in treatment, 25% of children with ALL develop recurrent disease. Conversely, another 25% of children who now receive dose intensification are likely “over-treated” and may well be cured using less intensive regimens resulting in fewer toxicities and long term side effects. Thus, a major challenge for the treatment of children with ALL in the next decade is to improve and refine ALL diagnosis and risk classification schemes in order to precisely tailor therapeutic approaches to the biology of the tumor and the genotype of the host.

Leukemia in the first 12 months of life (referred to as infant leukemia) is extremely rare in the United States, with about 150 infants diagnosed each year. There are several clinical and genetic factors that distinguish infant leukemia from acute leukemias that occur in older children. First, while the percentage of acute lymphoblastic leukemia (ALL) cases is far more frequent (approximately five times) than acute myeloid leukemia in children from ages 1-15 years, the frequency of ALL and AML in infants less than one year of age is approximately equivalent. Secondly, in contrast to the extensive heterogeneity in cytogenetic abnormalities and chromosomal rearrangements in older children with ALL and AML, nearly 60% of acute leukemias in infants have chromosomal rerrangments involving the MLL gene (for Mixed Lineage Leukemia) on chromosome 11 q23. MLL translocations characterize a subset of human acute leukemias with a decidedly unfavorable prognosis. Current estimates suggest that about 60% of infants with AML and about 80% of infants with ALL have a chromosomal rearrangment involving MLL abnormality in their leukemia cells. Whether hematopoietic cells in infants are more likely to undergo chromosomal rearrangements involving 11q13 or whether this 11q13 rearrangement reflects a unique environmental exposure or genetic susceptibliity remains to be determined.

The modern classification of acute leukemias in children and adults relies on morphologic and cytochemical features that may be useful in distinguishing AML from ALL, changes in the expression of cell surface antigens as a precursor cell differentiates, and the presence of specific recurrent cytogenetic or chromosomal rearrangements in leukemic cells. Using monoclonal antibodies, cell surface antigens (called clusters of differentiation (CD)) can be identified in cell populations; leukemias can be accurately classified by this means (immunophenotyping). By immunophenotyping, it is possible to classify ALL into the major categories of “common—CD10+ B-cell precursor” (around 50%), “pre-B” (around 25%), “T” (around 15%), “null” (around 9%) and “B” cell ALL (around 1%). All forms other than T-ALL are considered to be derived from some stage of B-precursor cell, and “null” ALL is sometimes referred to as “early B-precursor” ALL.

Current risk classification schemes for ALL in children from 1-18 years of age use clinical and laboratory parameters such as patient age, initial white blood cell count, and the presence of specific ALL-associated cytogenetic abnormalities to stratify patients into “low,” “standard,” “high,” and “very high” risk categories. National Cancer Institute (NCI) risk criteria are first applied to all children with ALL, dividing them into “NCI standard risk” (age 1.00-9.99 years, WBC<50,000) and “NCI high risk” (age>10 years, WBC>50,000) based on age and initial white blood cell count (WBC) at disease presentation. In addition to these general NCI risk criteria, classic cytogenetic analysis and molecular genetic detection of frequently recurring cytogenetic abnormalities have been used to stratify ALL patients more precisely into “low,” “standard,” “high,” and “very high” risk categories. Table 1A shows the 4-year event free survival (EFS) projected for each of these groups.

TABLE 1A
Recurrent Genetic Subtypes of B and T Cell ALL

| | Associated Genetic | Frequency | Risk |
|---|---|---|---|
| Subtype | Abnormalities | in Children | Category |
| B-Precursor | Hyperdiploid DNA Content; | 25% of B | Low |
| ALL | Trisomies of Chromosomes 4, | Precursor | |
| | 10, 17 | Cases | |
| | t(12; 21)(p13; q22): TEL/AML1 | 28% of B | Low |
| | | Precursor | |
| | | Cases | |
| | 11q23/MLL Rearrangements; | 4% of B | High |
| | particularly t(4; 11)(q21; q23) | Precursor | |
| | | Cases; | |
| | t(1; 19)9q23; p13) - E2A/PBX1 | >80% of | High |
| | | Infant ALL | |
| | t(9; 22)(q34; q11): BCR/ABL | 6% of B | Very |
| | | Precursor | High |
| | | Cases | |
| | Hypodiploidy | 2% of B | Very |
| | | Precursor | High |
| | | Cases | |
| | | Relatively | |
| | | Rare | |
| B-ALL | t(8; 14)(q24; q32) - IgH/MYC | 5% of all | High |
| | | B lineage | |
| | | ALL cases | |
| T-ALL | Numerous translocations | 7% of ALL | Not |
| | involving the TCR αβ (7q35) or | cases | Clearly |
| | TCR γδ (14q11) loci | | Defined |

The rate of disappearance of both B precursor and T ALL leukemic cells during induction chemotherapy (assessed morphologically or by other quantitative measures of residual disease) has also been used as an assessment of early therapeutic response and as a means of targeting children for therapeutic intensification (Gruhn et al., Leukemia 12:675-15 681, 1998; Foroni et al., Br. J. Haematol. 105:7-24, 1999; van Dongen et al., Lancet 352:1731-1738, 1998; Cave et al., N. Engl. J. Med. 339:591-598, 1998; Coustan-Smith et al., Lancet 351:550-554, 1998; Chessells et al., Lancet 343:143-148, 1995; Nachman et al., N. Engl. J. Med. 338:1663-1671, 1998).

Children with “low risk” disease (22% of all B precursor ALL cases) are defined as having standard NCI risk criteria, the presence of low risk cytogenetic abnormalities (t(12;21)/TEL;AML1 or trisomies of chromosomes 4 and 10), and a rapid early clearance of bone marrow blasts during induction chemotherapy. Children with “standard risk” disease (50% of ALL cases) are NCI standard risk without “low risk” or unfavorable cytogenetic features, or, are children with low risk cytogenetic features who have NCI high risk criteria or slow clearance of blasts during induction. Although therapeutic intensification has yielded significant improvements in outcome in the low and standard risk groups of ALL, it is likely that a significant number of these children are currently “over-treated” and could be cured with less intensive regimens resulting in fewer toxicities and long term side effects. Conversely, a significant number of children even in these good risk categories still relapse and a precise means to prospectively identify them has remained elusive. Nearly 30% of children with ALL have “high” or “very high” risk disease, defined by NCI high risk criteria and the presence of specific cytogenetic abnormalities (such as t(1;19), t(9;22) or hypodiploidy) (Table 1); again, precise measures to distinguish children more prone to relapse in this heterogeneous group have not been established.

Despite these efforts, current diagnosis and risk classification schemes remain imprecise. Children with ALL are more prone to relapse who require more intensive approaches and children with low risk disease who could be cured with less intensive therapies are not adequately predicted by current classification schemes and are distributed among all currently defined risk groups. Although pre-treatment clinical and tumor genetic stratification of patients has generally improved outcomes by optimizing therapy, variability in clinical course continues to exist among individuals within a single risk group and even among those with similar prognostic features. In fact, the most significant prognostic factors in childhood ALL explain no more than 4% of the variability in prognosis, suggesting that yet undiscovered molecular mechanisms dictate clinical behavior (Donadieu et al., Br J Haematol, 102:729-739, 1998). A precise means to prospectively identify such children has remained elusive.

With the advent of modern combination chemotherapy and transplantation, significant advances have been made in the treatment of the acute leukemias, particularly in children. Yet despite these advances, 23,000 of the more than 33,000 children and adults diagnosed with leukemia in 2005 will ultimately die of resistant or relapsed disease (1). The therapeutic advances that have been achieved in the acute leukemias, particularly in pediatric acute lymphoblastic leukemia (ALL), have come in part through the development of detailed risk classification schemes based on clinical features, the presence or absence of specific cytogenetic or molecular genetic abnormalities, and measures of early therapeutic response that may be used to tailor the choice of therapy and its intensity to a patient's relapse risk (2). Yet current risk classification schemes do not fully reflect the tremendous molecular heterogeneity of the acute leukemias and do not precisely identify those patients who are more prone to relapse, those who might be cured with less intensive regimens resulting in fewer toxicities and long term side effects, or those who will respond to newer targeted therapeutic agents. It has thus been our hypothesis that large scale genomic and proteomic technologies that measure global patterns of gene expression in leukemic cells will yield systematic profiles that can be used to improve outcome prediction, risk classification, and therapeutic targeting in the acute leukemias. Previously funded under the NCI Director's Challenge Program: Toward a Molecular Classification of Tumors (NCI CA88361-PI: CL Willman; NCI CA85053-PI: JP Radich), we have worked with two of the National Cancer Institute (NCI) Cooperative Oncology Groups (the Children's Oncology Group or COG and the Southwest Oncology Group or SWOG) to design retrospective patient cohorts from which we derived rigorously cross-validated gene expression profiles in both children and adults with acute leukemia. Over the past four years, we have built highly collaborative multidisciplinary laboratory, statistical, and computational teams; developed reproducible and sensitive methods for performing gene expression arrays; designed data warehouses for storage of large gene expression datasets fully annotated with clinical, outcome, and experimental information; and developed and applied robust statistical and computational methods and novel visualization tools for array data analysis. The results of these analyses are now published, in press, or submitted for publication (3-19) and our fully annotated gene expression and clinical datasets are publicly available at the NCI Gene Expression Data Portal website (http://gedp.nci.nih.gov/dc).

The major scientific challenge in pediatric ALL is to improve risk classification schemes and outcome prediction in order to: 1) identify those children who are most likely to relapse who require intensive or novel regimens for cure; and 2) identify those children who can be cured with less intensive regimens with fewer toxicities and long term side effects. In contrast to pediatric ALL, overall outcome in adult ALL remains poor and risk classification schemes are rarely employed. Thus, any new advances in molecular classification and outcome prediction would have a significant clinical impact. To begin to identify new genes that could improve molecular classification, outcome prediction, and therapeutic targeting in ALL, and, to develop leads for potential genes and pathways that could be exploited for the development of new therapies, we recently obtained comprehensive gene expression profiles in a retrospective case control study of 254 children with ALL registered to NCI-sponsored clinical trials.

### BRIEF DESCRIPTION OF THE FIGURES

FIG. 1A shows a modeling of the most predominant network and pathway from the list of 26 genes in the pediatric ALL gene expression classifier (Table 1), using Ingenuity Pathways Analysis (see ingenuity.com). This cell death pathway was the most prominent network, including 8 genes from the classifier (Table 1). These 8 genes are listed in the text; they are networked with other genes in this pathway (noted with a white background)

FIG. 1B shows a pathway model of the CCL5 (RANTES)/CD44 pathway involving 7 genes from the classifier (these genes are noted in bolded capital letters).

FIG. 2 shows the expression of the 26 Predictive Genes in the UNM ALL Cohort scored by the predictive outcome model. Patients were divided into higher, medium, and low risk groups based on CCR score. Darker areas indicate relatively high expression levels, whereas lighter areas indicate relatively low expression levels.

FIG. 3 shows the expression of 9 gene regression model predicting CCR rate. Note that the expression of six (6) genes are up and are predictive of CCR, whereas the expression of three (3) genes are up and predictive of therapeutic failure.

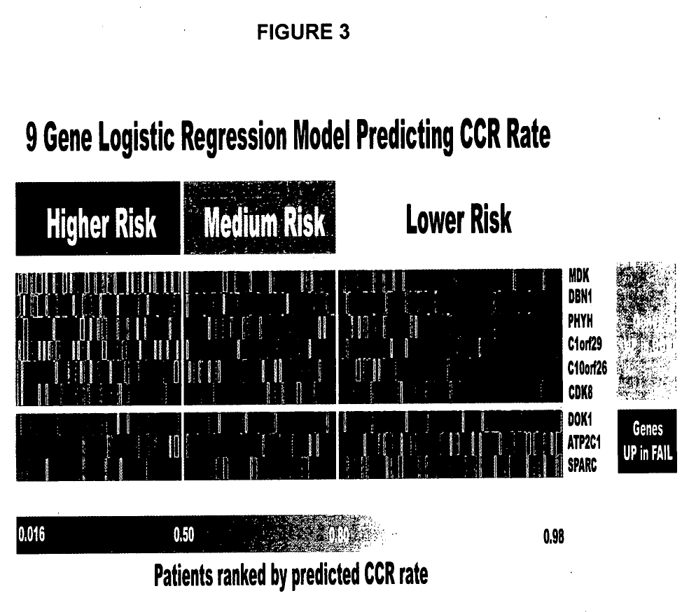

FIG. 4 shows the distribution of outcome scores and expression of the predictive genes in an independent cohort of pediatric ALL cases as discussed hereinbelow.

### BRIEF DESCRIPTION OF THE INVENTION

The present invention evaluates a gene expression profile and identifies prognostic genes of cancers, in particular leukemia, more particularly B-precursor acute lymphoblastic leukemia (ALL). The present invention provides a method of determining prognosis of leukemia, in particular, acute lymphoblastic leukemia, more particularly B-precursor ALL and predicting therapeutic outcome of a patient. The method comprises the steps of first establishing the threshold value of at least three prognostic genes of leukemia, preferably at least eight prognostic genes, or preferably, as many as 26 prognostic genes. Then, the amount of the prognostic gene(s) from a patient inflicted of leukemia is determined. The amount of the prognostic gene present in that patient is compared with the established threshold value of the prognostic gene(s) which is indicative of therapeutic success or failure, whereby the prognostic outcome of the patient is determined.

In certain embodiments, the amount of the prognostic gene is determined by the quantitation of a transcript encoding the sequence of the prognostic gene; or a polypeptide encoded by the transcript. The quantitation of the transcript can be based on hybridization to the transcript. The quantitation of the polypeptide can be based on antibody detection. The method optionally comprises a step of amplifying nucleic acids from the tissue sample before the evaluating (pcr analysis). In a number of embodiments, the evaluating is of a plurality of prognostic genes, preferably at least three prognostic genes, and more preferably at least eight genes as otherwise described herein, preferably as many as 26 genes. The prognosis contributes to selection of a therapeutic strategy, which may be traditional therapy for B-precursor ALL, or a more aggressive therapy based upon a traditional therapy or non-traditional therapy.

The present invention is directed to methods for outcome prediction and risk classification in leukemia, especially B precursor acute lymphoblastic leukemia (ALL). In one embodiment, the invention provides a method for classifying leukemia in a patient that includes obtaining a biological sample from a patient; determining the expression level for a selected gene product, more preferably a group of selected gene products to yield an observed gene expression level; and comparing the observed gene expression level for the selected gene product(s) to control gene expression levels. The control gene expression level can be the expression level observed for the gene product(s) in a control sample, or a predetermined expression level for the gene product. An observed expression level (higher or lower) that differs from the control gene expression level is indicative of a disease classification. In another aspect, the method can include determining a gene expression profile for selected gene products in the biological sample to yield an observed gene expression profile; and comparing the observed gene expression profile for the selected gene products to a control gene expression profile for the selected gene products that correlates with a disease classification, for example ALL, and in particular B precursor ALL; wherein a similarity between the observed gene expression profile and the control gene expression profile is indicative of the disease classification.

The disease classification can be, for example, a classification preferably based on predicted outcome (remission vs therapeutic failure); a classification based upon clinical characteristics of patients, a classification based on karyotype; a classification based on leukemia subtype; or a classification based on disease etiology. Where the classification is based on disease outcome, the observed gene product is preferably a gene product selected from at least three of the following group of eight gene products, more preferably four, five, six, seven, or more preferably all eight gene products: midkine (neurite growth-promoting factor 2), CHST 10 (carbohydrate sulfotransferase 1or HNK1-sulfotransferase), PHYN (phytanoyl-CoA hydroxylase), IF144L (Interferon-induced protein 44-like, C1 or f29), OPAL 1, CDK8 (cyclin-dependent kinase 8), DOK1 (docking protein 1-62kD and downstream of tyrosine kinase 1) and ATP2C1 (ATPase-Ca++ transporting type 2C member 1). Alternatively, the invention may rely on measuring the previous eight gene products or those eight gene products in addition to at least one or more of the other gene products within a longer list of 26 gene products which appears in Table 1, below. Measurement of all 26 gene products set forth in Table 1, below, may also be performed to provide an accurate assessment of therapeutic intervention.

The invention further provides for a method for predicting therapeutic outcome in a B precursor ALL leukemia patient that includes obtaining a biological sample from a patient; determining the expression level for selected gene products associated with outcome to yield an observed gene expression level; and comparing the observed gene expression level for the selected gene product(s) to a control gene expression level for the selected gene product. The control gene expression level for the selected gene product can include the gene expression level for the selected gene product observed in a control sample, or a predetermined gene expression level for the selected gene product; wherein an observed expression level that is different from the control gene expression level for the selected gene product(s) is indicative of predicted remission. The method may determine gene expression levels of at least three gene products selected from the group consisting of midkine (neurite growth-promoting factor 2), CHST 10 (carbohydrate sulfotransferase 1or HNK1-sulfotransferase), PHYN (phytanoyl-CoA hydroxylase), IF144L (Interferon-induced protein 44-like, C1orf29), OPAL 1, CDK8 (cyclin-dependent kinase 8), DOK1 (docking protein 1-62 kD and downstream of tyrosine kinase 1) and ATP2C1 (ATPase-Ca++ transporting type 2C member 1), more preferably at least four of the above gene products, more preferably at least five of the above gene products, more preferably at least six of the above gene products, even more preferably seven of the above gene products and most preferably all eight of the above gene products. In addition to the above eight products, the present method may also include measuring expression of at least one additional gene product and up to 18 additional gene products according to Table 1. In certain preferred aspects of the invention, the expression levels of all 26 gene products (Table 1) may be determined and compared to a predetermined gene expression level, wherein a measurement above or below a predetermined expression level is indicative of the likelihood of a favorable therapeutic response (continuous complete remission or CCR) or therapeutic failure. In the case where therapeutic failure is predicted, the use of more aggressive protocols of traditional anti-cancer therapies (higher doses and/or longer duration of drug administration) or experimental therapies may be advisable.

Optionally, the method further comprises determining the expression level for other gene products within the list of gene products and comparing in a similar fashion the observed gene expression levels for the selected gene products with a control gene expression level for those gene products, wherein an observed expression level for these gene products that is different from (above or below) the control gene expression level for that gene product is further indicative of predicted remission.

The invention further includes a method for treating leukemia comprising administering to a leukemia patient a therapeutic agent that modulates the amount or activity of the gene product(s) associated with outcome, in particular, midkine (neurite growth-promoting factor 2), CHST 10 (carbohydrate sulfotransferase 1 or HNK1 -sulfotransferase), PHYN (phytanoyl-CoA hydroxylase), IF144L (Interferon-induced protein 44-like, C1orf29), OPAL 1, CDK8 (cyclin-dependent kinase 8), DOK1 (docking protein 1-62 kD and downstream of tyrosine kinase 1) and ATP2C 1 (ATPase-Ca++ transporting type 2C member 1). Preferably, the method modulates at least three of the eight gene products as set forth above, four of the gene products, five of the gene products, six of the gene products, seven of the gene products or eight of the gene products, set forth above to a level above or below a predetermined expression level (see Table 1). It is noted that the expression level of midkine (neurite growth-promoting factor 2), CHST 10 (carbohydrate sulfotransferase 1 or HNK1-sulfotransferase), PHYN (phytanoyl-CoA hydroxylase), IF144L (Interferon-induced protein 44-like, C1orf29), OPAL 1 and CDK8 (cyclin-dependent kinase 8) in a patient which is above a predetermined value (high expression) is generally indicative of a favorable therapeutic outcome, whereas the expression level of DOK1 (docking protein 1-62 kD and downstream of tyrosine kinase 1) and ATP2C1 (ATPase-Ca++ transporting type 2C member 1) which is above a predetermined value is generally indicative of a failed therapeutic outcome and below a predetermined value is generally indicative of a favorable therapeutic outcome (low expression).

Also provided by the invention is an in vitro method for screening a compound useful for treating leukemia. The invention further provides an in vivo method for evaluating a compound for use in treating leukemia. The candidate compounds are evaluated for their effect on the expression level(s) of one or more gene products associated with outcome in leukemia patients, preferably at least three of those gene products, four of those gene products, five of those gene products, six of those gene products, seven of those gene products, and preferably eight of those eight gene products. In certain preferred instances, the expression levels of the above eight gene products and at least one additional gene product, up to 18 additional gene products as set forth in table 1 hereof may be measured to determine a therapeutic outcome.

The preferred eight gene products are as identified for example, using probe sets (9 gene probes identifying the 8 gene products, noted in Table 1, columns 1 by the (*), include: MIDKINE (two probe sets), CHST10 (HNK-1 sulfotransferase), PHYN, IFI44L (C1orf29), OPAL1, CDK8, DOK1, and ATP2C 1. These 8 genes and their expression above or below a predetermined expression level (Table 1) were more predictive of overall outcome than the traditional variables currently used for risk classification in pediatric ALL (p<0.0001). This predictive model was then tested in an independent cohort of pediatric ALL cases (20) and was found to predict outcome with extremely high statistical significance (p-value<1.0⁻⁸). It is noted that the expression of gene products of the eight genes listed above, as well as at least one additional gene from the list appearing in Table 1 and in certain preferred instances, the expression of all 26 gene products of Table 1 may be measured and compared to predetermined expression levels to provide the greater degrees of certainty of a therapeutic outcome.

### DETAILED DESCRIPTION OF THE INVENTION

Gene expression profiling can provide insights into disease etiology and genetic progression, and can also provide tools for more comprehensive molecular diagnosis and therapeutic targeting. The biologic clusters and associated gene profiles identified herein may be useful for refined molecular classification of acute leukemias as well as improved risk assessment and classification, especially of acute lymphoblastic leukemia (ALL), such as particular B precursor ALL (especially pediatric B precursor ALL). In addition, the invention has identified numerous genes, including but not limited to the genes midkine (neurite growth-promoting factor 2), CHST 10 (carbohydrate sulfotransferase 1 or HNK1-sulfotransferase), PHYN (phytanoyl-CoA hydroxylase), IF144L (Interferon-induced protein 44-like, C1orf29), OPAL1, CDK8 (cyclin-dependent kinase 8), DOK1 (docking protein 1-62 kD and downstream of tyrosine kinase 1) and ATP2C1 (ATPase-Ca++ transporting type 2C member 1) as well as numerous additional genes which are presented in Table 1 hereof, that are, alone or in combination, strongly predictive of therapeutic outcome in pediatric ALL, and in particular B precursor ALL. The genes identified herein, and the gene products from said genes, including proteins they encode, can be used to refine risk classification and diagnostics, to make outcome predictions and improve prognostics, and to serve as therapeutic targets in infant leukemia and pediatric ALL, especially B-precursor ALL.

“Gene expression” as the term is used herein refers to the production of a biological product encoded by a nucleic acid sequence, such as a gene sequence. This biological product, referred to herein as a “gene product,” may be a nucleic acid or a polypeptide. The nucleic acid is typically an RNA molecule which is produced as a transcript from the gene sequence. The RNA molecule can be any type of RNA molecule, whether either before (e.g., precursor RNA) or after (e.g., mRNA) post-transcriptional processing. cDNA prepared from the mRNA of a sample is also considered a gene product. The polypeptide gene product is a peptide or protein that is encoded by the coding region of the gene, and is produced during the process of translation of the mRNA.

The term “gene expression level” refers to a measure of a gene product(s) of the gene and typically refers to the relative or absolute amount or activity of the gene product.

The term “gene expression profile” as used herein is defined as the expression level of two or more genes. The term gene includes all natural variants of the gene. Typically a gene expression profile includes expression levels for the products of multiple genes in given sample, up to 13,000 in the experiments described herein, preferably determined using an oligonucleotide microarray.

Unless otherwise specified, “a,” “an,” “the,” and “at least one” are used interchangeably and mean one or more than one.

The term “patient” shall mean within context an animal, preferably a mammal, more preferably a human patient, more preferably a human child who is undergoing or will undergo therapy or treatment for leukemia, especially B-precursor acute lymphoblastic leukemia.

The term “traditional therapy” relates to therapy (protocol) which is typically used to treat, leukemia, for example, B-precursor ALL (especially pediatric) and can include Memorial Sloan-Kettering New York II therapy (NY II), UKALLR2, AL 841, AL851, ALHR88, MCP841 (India), as well as modified BFM (Berlin-Frankfurt-Münster) therapy, BMF-95 or other therapy, including ALinC 17 therapy as is well-known in the art. In the present invention the term “more aggressive therapy” or “alternative therapy” usually means a more aggressive version of conventional therapy typically used to treat leukemia, for example pediatric B-precursor ALL, using for example, conventional or traditional chemotherapeutic agents at higher dosages and/or for longer periods of time in order to increase the likelihood of a favorable therapeutic outcome. It may also refer, in context, to experimental therapies for treating leukemia, rather than simply more aggressive versions of conventional (traditional) therapy.

Diagnosis, Prognosis and Risk Classification

Current parameters used for diagnosis, prognosis and risk classification in pediatric ALL are related to clinical data, cytogenetics and response to treatment. They include age and white blood count, cytogenetics, the presence or absence of minimal residual disease (MRD), and a morphological assessment of early response (measured as slow or rapid early therapeutic response). As noted above however, these parameters are not always well correlated with outcome, nor are they precisely predictive at diagnosis.

Prognosis is typically recognized as a forecast of the probable course and outcome of a disease. As such, it involves inputs of both statistical probability, requiring numbers of samples, and outcome data. In the present invention, outcome data is utilized in the form of continuous complete remission (CCR) of ALL or therapeutic failure (non-CCR). A patient population of hundreds is included, providing statistical power.

The ability to determine which cases of leukemia, especially B precursor acute lymphoblastic leukemia will respond to treatment, and to which type of treatment, would be useful in appropriate allocation of treatment resources. It would also provide guidance as to the aggressiveness of therapy in producing a favorable outcome (continuous complete remission or CCR). As indicated above, the various standard therapies have significantly different risks and potential side effects, especially therapies which are more aggressive or even experimental in nature. Accurate prognosis would also minimize application of treatment regimens which have low likelihood of success and would allow a more efficient aggressive or even an experimental protocol to be used without wasting effort on therapies unlikely to produce a favorable therapeutic outcome, preferably a continuous complete remission. Such also could avoid delay of the application of alternative treatments which may have higher likelihoods of success for a particular presented case. Thus, the ability to evaluate individual leukemia cases, especially B-precursor acute lymphoblastic leukemia, for markers which subset into responsive and non-responsive groups for particular treatments is very useful.

Current models of leukemia classification are poor at distinguishing between cancers that have similar histopathological features but vary in clinical course and outcome. Identification of novel prognostic molecular markers is a priority if radical treatment is to be offered on a more selective basis to those leukemia patients with disease states which do not respond favorably to conventional therapy. A novel strategy is described to discover/assess/measure molecular markers for leukemia by assessing gene expression in leukemia patients and modeling these data based on a predetermined gene product expression for numerous patients having a known clinical outcome. The invention herein is directed to defining different forms of leukemia, particularly, B-precursor acute lymphoblastic leukemia, especially pediatric B-precursor acute lymphoblastic leukemia, by measuring expression gene products which can translate directly into therapeutic prognosis. Such prognosis allows for application of a treatment regimen having a greater statistical likelihood of cost effective treatments and minimization of negative side effects from the different/various treatment options.

In preferred aspects, the present invention provides an improved method for identifying and/or classifying acute leukemias, especially pediatric B precursor ALL and for providing an indication of the therapeutic outcome of the patient based upon an assessment of expression levels of particular genes. Expression levels are determined for one or more genes associated with outcome, risk assessment or classification, karyotpe (e.g., MLL translocation) or subtype (e.g., ALL vs. AML; pre-B ALL vs. T-ALL. Genes that are particularly relevant for diagnosis, prognosis and risk classification, especially for pediatric B precursor ALL, according to the invention include those described in the tables (especially Table 1) and figures herein. The gene expression levels for the gene(s) of interest in a biological sample from a patient diagnosed with or suspected of having an acute leukemia, especially B precursor ALL are compared to gene expression levels observed for a control sample, or with a predetermined gene expression level. Observed expression levels that are higher or lower than the expression levels observed for the gene(s) of interest in the control sample or that are higher or lower than the predetermined expression levels for the gene(s) of interest (as set forth in Table 1) provide information about the acute leukemia that facilitates diagnosis, prognosis, and/or risk classification and can aid in treatment decisions, especially whether to use a more of less aggressive therapeutic regimen or perhaps even an experimental therapy. When the expression levels of multiple genes are assessed for a single biological sample, a gene expression profile is produced.

In one aspect, the invention provides genes and gene expression profiles that are correlated with outcome (i.e., complete continuous remission vs. therapeutic failure) in leukemia, especially ALL, and in particular B precursor all, especially pediatric B precursor. Assessment of one or more of these genes according to the invention, preferably at least three and more preferably eight of these genes as otherwise described herein, or as many as 26 genes as set forth in Table 1 in a given gene profile can be integrated into revised risk classification schemes, therapeutic targeting and clinical trial design. In one embodiment, the expression levels of a particular gene (gene products) are measured, and that measurement is used, either alone or with other parameters, to assign the patient to a particular risk category. The invention identifies several genes whose expression levels, either alone or in combination, are associated with outcome, including but not limited to at least three genes and preferably all eight genes selected from the group consisting of midkine (neurite growth-promoting factor 2), CHST 10 (carbohydrate sulfotransferase 1 or HNK1-sulfotransferase), PHYN (phytanoyl-CoA hydroxylase), IF 144L (Interferon-induced protein 44-like, C1orf29), OPAL 1, CDK8 (cyclin-dependent kinase 8), DOK1 (docking protein 1-62 kD and downstream of tyrosine kinase 1) and ATP2C 1 (ATPase-Ca++ transporting type 2C member 1); and the remaining genes listed in Table 1.

Some of these genes (e.g., OPAL1 and several others as listed above and in Table 1, below) exhibit a positive association between expression level and outcome. For these genes, expression levels above a predetermined threshold level (or higher than that exhibited by a control sample) is predictive of a positive outcome (continuous complete remission). Our data suggests that direct measurement of the expression level of at least three of these genes, preferably at least including OPAL1, more preferably all eight of these genes, can be used in refining risk classification and outcome prediction in pediatric ALL, especially B precursor ALL. In particular, it is expected such measurements can be used to refine risk classification in children who are otherwise classified as having low risk ALL, as well as to precisely identify children with high risk ALL who could be cured with less intensive therapies.

OPAL1, in particular, is a strong predictor for outcome, therefore in preferred aspects, the expression of at least three genes selected from the group consisting of the eight genes cited below, preferably including OPAL 1, is measured. In preferred aspects the expression of all eight genes is measured. Expression levels for multiple genes can be measured. For example, if normalized expression levels for midkine (neurite growth-promoting factor 2), CHST 10 (carbohydrate sulfotransferase 1 or HNK1-sulfotransferase), PHYN (phytanoyl-CoA hydroxylase), IF144L (Interferon-induced protein 44-like, C1orf29), OPAL 1, CDK8 (cyclin-dependent kinase 8) are higher than a predetermined value and normalized expression levels for DOK1 (docking protein 1-62 kD and downstream of tyrosine kinase 1) and ATP2C 1 (ATPase-Ca++ transporting type 2C member 1) are lower then a predetermined value (higher expression levels of these genes are predictive of therapeutic failure), a favorable outcome can be predicted with greater certainty. Optionally, expression levels of sparc (secreted protein, acidic, cysteine-rich; osteonectin) may also be measured and included within the analysis. Alternatively, in addition to the eight genes which are measured as described above, the expression of all eight genes described above and one or more additional genes (as set forth in Table 1) can be measured and used for predicting therapeutic outcome. In certain aspects of the invention where very high reliability is desired/required, the expression levels of all 26 genes (as per Table 1) may be measured and compared with a predetermined value such that a measurement above or below the predetermined value of expression for the group of genes is indicative of a favorable therapeutic outcome (continuous complete remission) or a therapeutic failure. In the event of a predictive favorable therapeutic outcome, convention anti-cancer therapy may be used and in the event of a predictive unfavorable outcome (failure), more aggressive therapy may be recommended and implemented.

The expression levels of multiple (two or more, preferably three or more, more preferably eight as described hereinabove and in addition to the eight, up to twenty-six within the genes listed in Table 1) in one or more lists of genes associated with outcome can be measured, and those measurements are used, either alone or with other parameters, to assign the patient to a particular risk category as it relates to a predicted therapeutic outcome. For example, gene expression levels of multiple genes can be measured for a patient (as by evaluating gene expression using an Affymetrix microarray chip) and compared to a list of genes whose expression levels (high or low) are associated with a positive (or negative) outcome. If the gene expression profile of the patient is similar to that of the list of genes associated with outcome, then the patient can be assigned to a low (or high, as the case may be) risk category. The correlation between gene expression profiles and class distinction can be determined using a variety of methods. Methods of defining classes and classifying samples are described, for example, in Golub et al, U.S. patent application Publication No. 2003/0017481 published Jan. 23, 2003, and Golub et al., U.S. patent application Publication No. 2003/0134300, published Jul. 17, 2003. The information provided by the present invention, alone or in conjunction with other test results, aids in sample classification and diagnosis of disease.

Computational analysis using the gene lists and other data, such as measures of statistical significance, as described herein is readily performed on a computer. The invention should therefore be understood to encompass machine readable media comprising any of the data, including gene lists, described herein. The invention further includes an apparatus that includes a computer comprising such data and an output device such as a monitor or printer for evaluating the results of computational analysis performed using such data.

In another aspect, the invention provides genes and gene expression profiles that are correlated with cytogenetics. This allows discrimination among the various karyotypes, such as MLL translocations or numerical imbalances such as hyperdiploidy or hypodiploidy, which are useful in risk assessment and outcome prediction.

In yet another aspect, the invention provides genes and gene expression profiles that are correlated with intrinsic disease biology and/or etiology. In other words, gene expression profiles that are common or shared among individual leukemia cases in different patients can be used to define intrinsically related groups (often referred to as clusters) of acute leukemia that cannot be appreciated or diagnosed using standard means such as morphology, immunophenotype, or cytogenetics. Mathematical modeling of the very sharp peak in ALL incidence seen in children 2-3 years old (>80 cases per million) has suggested that ALL may arise from two primary events, the first of which occurs in utero and the second after birth (Linet et al., Descriptive epidemiology of the leukemias, in Leukemias, 5^{th} Edition. ES Henderson et al. (eds). WB Saunders, Philadelphia. 1990). Interestingly, the detection of certain ALL-associated genetic abnormalities in cord blood samples taken at birth from children who are ultimately affected by disease supports this hypothesis (Gale et al., Proc. Natl. Acad. Sci. U.S.A., 94:13950-13954, 1997; Ford et al., Proc. Natl. Acad. Sci. U.S.A., 95:4584-4588, 1998).

The results for pediatric B precursor ALL suggest that this disease is composed of novel intrinsic biologic clusters defined by shared gene expression profiles, and that these intrinsic subsets cannot reliably be defined or predicted by traditional labels currently used for risk classification or by the presence or absence of specific cytogenetic abnormalities. We have identified 8 genes for ALL, in particular B precursor ALL using the methods set forth hereinbelow, for identifying candidate genes associated with classification and outcome. These 8 genes are positive predictors of favorable outcome in B precursor ALL patients. In addition, the present invention has identified eight genes (the last eight genes in Table 1) which correlate with failed therapy. Thus, a measurement of the expression of these eight genes which is higher than predetermined values for each of these genes is predictive of a high likelihood of a therapeutic failure using traditional B precursor ALL therapies. High expression for these eight genes would dictate an early aggressive therapy or experimental therapy in order to increase the likelihood of a favorable therapeutic outcome.

Some genes in these clusters are metabolically related, suggesting that a metabolic pathway that is associated with cancer initiation or progression. Other genes in these metabolic pathways, like the genes described herein but upstream or downstream from them in the metabolic pathway, thus can also serve as therapeutic targets.

In yet another aspect, the invention provides genes and gene expression profiles which may be used to discriminate acute myeloid leukemia (AML) from acute lymphoblastic leukemia (ALL) in infant leukemias by measuring the expression levels of the gene product(s) correlated with ALL or AML, especially B-precursor ALL.

Another aspect of the invention provides genes and gene expression profiles that discriminate pre-B lineage ALL B-precursor ALL) from T ALL in pediatric leukemias by measuring expression levels of gene product(s) correlated with pre-B lineage ALL or T ALL.

It should be appreciated that while the present invention is described primarily in terms of human disease, it is useful for diagnostic and prognostic applications in other mammals as well, particularly in veterinary applications such as those related to the treatment of acute leukemia in cats, dogs, cows, pigs, horses and rabbits.

Further, the invention provides methods for computational and statistical methods for identifying genes, lists of genes and gene expression profiles associated with outcome, karyotype, disease subtype and the like as described herein.

In sum, the present invention has identified a group of genes which strongly correlate with favorable/unfavorable outcome in B precursor acute lymphoblastic leukemia and contribute unique information to allow the reliable prediction of a therapeutic outcome in B precursor ALL.

Measurement of Gene Expression Levels

Gene expression levels are determined by measuring the amount or activity of a desired gene product (i.e., an RNA or a polypeptide encoded by the coding sequence of the gene) in a biological sample. Any biological sample can be analyzed. Preferably the biological sample is a bodily tissue or fluid, more preferably it is a bodily fluid such as blood, serum, plasma, urine, bone marrow, lymphatic fluid, and CNS or spinal fluid. Preferably, samples containing mononuclear bloods cells and/or bone marrow fluids and tissues are used. In embodiments of the method of the invention practiced in cell culture (such as methods for screening compounds to identify therapeutic agents), the biological sample can be whole or lysed cells from the cell culture or the cell supernatant.

Gene expression levels can be assayed qualitatively or quantitatively. The level of a gene product is measured or estimated in a sample either directly (e.g., by determining or estimating absolute level of the gene product) or relatively (e.g., by comparing the observed expression level to a gene expression level of another samples or set of samples). Measurements of gene expression levels may, but need not, include a normalization process.

Typically, mRNA levels (or cDNA prepared from such mRNA) are assayed to determine gene expression levels. Methods to detect gene expression levels include Northern blot analysis (e.g., Harada et al., Cell 63:303-312 (1990)), S1 nuclease mapping (e.g., Fujita et al., Cell 49:357-367 (1987)), polymerase chain reaction (PCR), reverse transcription in combination with the polymerase chain reaction (RT-PCR) (e.g., Example III; see also Makino et al., Technique 2:295-301(1990)), and reverse transcription in combination with the ligase chain reaction (RT-LCR). Multiplexed methods that allow the measurement of expression levels for many genes simultaneously are preferred, particularly in embodiments involving methods based on gene expression profiles comprising multiple genes. In a preferred embodiment, gene expression is measured using an oligonucleotide microarray, such as a DNA microchip. DNA microchips contain oligonucleotide probes affixed to a solid substrate, and are useful for screening a large number of samples for gene expression. DNA microchips comprising DNA probes for binding polynucleotide gene products (mRNA) of the various genes from Table 1 are additional aspects of the present invention.

Alternatively or in addition, polypeptide levels can be assayed. Immunological techniques that involve antibody binding, such as enzyme linked immunosorbent assay (ELISA) and radioimmunoassay (RIA), are typically employed. Where activity assays are available, the activity of a polypeptide of interest can be assayed directly.

As discussed above, the expression levels of these markers in a biological sample may be evaluated by many methods. They may be evaluated for RNA expression levels. Hybridization methods are typically used, and may take the form of a PCR or related amplification method. Alternatively, a number of qualitative or quantitative hybridization methods may be used, typically with some standard of comparison, e.g., actin message. Alternatively, measurement of protein levels may performed by many means. Typically, antibody based methods are used, e.g., ELISA, radioimmunoassay, etc., which may not require isolation of the specific marker from other proteins. Other means for evaluation of expression levels may be applied. Antibody purification may be performed, though separation of protein from others, and evaluation of specific bands or peaks on protein separation may provide the same results. Thus, e.g., mass spectroscopy of a protein sample may indicate that quantitation of a particular peak will allow detection of the corresponding gene product. Multidimensional protein separations may provide for quantitation of specific purified entities.

The observed expression levels for the gene(s) of interest are evaluated to determine whether they provide diagnostic or prognostic information for the leukemia being analyzed. The evaluation typically involves a comparison between observed gene expression levels and either a predetermined gene expression level or threshold value, or a gene expression level that characterizes a control sample (“predetermined value”). The control sample can be a sample obtained from a normal (i.e., non-leukemic patient) or it can be a sample obtained from a patient with a known leukemia. For example, if a cytogenic classification is desired, the biological sample can be interrogated for the expression level of a gene correlated with the cytogenic abnormality, then compared with the expression level of the same gene in a patient known to have the cytogenetic abnormality (or an average expression level for the gene that characterizes that population).

The present study provides specific identification of multiple genes whose expression levels in biological samples will serve as markers to evaluate leukemia cases. These markers have been selected for statistical correlation to disease outcome data on a large number of leukemia patients.

Treatment of Infant Leukemia and Pediatric B-Precursor ALL

The genes identified herein that are associated with outcome of a disease state may provide insight into a treatment regimen. That regimen may be that traditionally used for the treatment of leukemia (as discussed hereinabove) in the case where the analysis of gene products from samples taken from the patient predicts a favorable therapeutic outcome, or alternatively, the chosen regimen may be a more aggressive approach (higher dosages of traditional therapies for longer periods of time) or even experimental therapies in instances where the predictive outcome is that of failure of therapy.

In addition, the present invention may provide new treatment methods, agents and regimens for the treatment of leukemia, including B-precursor acute lymphoblastic leukemia, especially pediatric B-precursor ALL. The genes identified herein that are associated with outcome and/or specific disease subtypes or karyotypes are likely to have a specific role in the disease condition, and hence represent novel therapeutic targets. Thus, another aspect of the invention involves treating infant leukemia and pediatric ALL patients by modulating the expression of one or more genes described herein to a desired expression level as described in Table 1.

In the case of those gene products (Table 1) whose increased or decreased expression (whether above or below a predetermined value) is associated with a favorable outcome or failure, the treatment method of the invention will involve enhancing the expression of those gene products in which a favorable therapeutic outcome is predicted by such enhancement and inhibiting the expression of those gene products in which enhanced expression is associated with failed therapy.

Thus, in the case of OPAL1 or other gene products such as midkine (neurite growth-promoting factor 2), CHST 10 (carbohydrate sulfotransferase 1 or HNK1-sulfotransferase), PHYN (phytanoyl-CoA hydroxylase), IFI44L (Interferon-induced protein 44-like, C1orf29) and CDK8 (cyclin-dependent kinase 8), increased expression of at least three and preferably all of these genes will be a therapeutic goal because enhanced expression of these genes together is predictive of a favorable therapeutic outcome and in the case of DOK1 (docking protein 1-62 kD and downstream of tyrosine kinase 1) and ATP2C 1 (ATPase-Ca++ transporting type 2C member 1) decreased expression is the goal as high expression of these two genes is a predictor of therapeutic failure. The same is true for the expression products of the other genes in the list which are found in Table 1—those which exhibit a favorable therapeutic outcome for high expression will be enhanced as a therapeutic goal, whereas as those which exhibit a failed therapeutic outcome for high expression will be inhibited as a therapeutic goal.

Thus, in the case of the 26 genes from Table 1, the increased or decreased expression levels for a particular gene as indicated in the table becomes a therapeutic goal in the treatment of leukemia, especially B-precursor ALL (especially pediatric B-precursor ALL). Therapeutic agents for effecting the increased or decreased expression levels may be identified and used as alternative therapies to traditional treatment modalities for leukemia, especially B-precursor ALL and either the increased or decreased expression of each of these genes will become a therapeutic goal for the treatment of cancer or the development of agents for the treatment of cancer. Thus, in this aspect of the present invention, especially in B precursor ALL (pediatric), the treatment method of the invention involves enhancing or inhibiting OPAL1 or other gene product expression as such gene expression is described in Table 1 with a therapeutic outcome. In preferred aspects, the therapeutic method preferably enhances expression of the genes OPAL1 midkine (neurite growth-promoting factor 2), CHST 10 (carbohydrate sulfotransferase 1 or HNK1-sulfotransferase), PHYN (phytanoyl-CoA hydroxylase), IFI44L (Interferon-induced protein 44-like, C1orf29) and CDK8 (cyclin-dependent kinase 8) and inhibits expression of DOK1 (docking protein 1-62 kD and downstream of tyrosine kinase 1) and ATP2C1 (ATPase-Ca++ transporting type 2C member 1). In addition to these eight genes, expression of at least one additional gene and preferably as many as 18 additional genes from the list in Table 1 (high expression CCR is desirable, low expression of failure is desirable) can be influenced to provide alternative therapies and anti-cancer agents.

For a number of the gene products identified herein, as above, increased expression is correlated with positive outcomes in leukemia patients. Thus, the invention includes a method for treating leukemia, such as infant leukemia and/or pediatric ALL, especially B precursor ALL (especially pediatric ALL) that involves administering to a patient a therapeutic agent that causes an increase in the amount or activity of OPAL I, and/or other polypeptides of interest where high expression has been identified herein to be positively correlated with favorable outcome (CCR, see Table 1). Preferably the increase in amount or activity of the selected gene product is at least about 10%, preferably 25%, most preferably 100% above the expression level observed in the patient prior to treatment.

The therapeutic agent can be a polypeptide having the biological activity of the polypeptide of interest (e.g., an OPAL1 or other gene product) or a biologically active subunit or analog thereof. Alternatively, the therapeutic agent can be a ligand (e.g., a small non-peptide molecule, a peptide, a peptidomimetic compound, an antibody, or the like) that agonizes (i.e., increases) the activity of the polypeptide of interest. For example, in the case of OPAL1 or other gene product, which is postulated to be a membrane-bound protein that may function as a receptor or signaling molecule, the invention encompasses the use of a proline-rich ligand of the WW-binding protein 1 to agonize OPAL1/G0 activity.

Gene therapies can also be used to increase the amount of a polypeptide of interest in a host cell of a patient. Polynucleotides operably encoding the polypeptide of interest can be delivered to a patient either as “naked DNA” or as part of an expression vector. The term vector includes, but is not limited to, plasmid vectors, cosmid vectors, artificial chromosome vectors, or, in some aspects of the invention, viral vectors. Examples of viral vectors include adenovirus, herpes simplex virus (HSV), alphavirus, simian virus 40, picomavirus, vaccinia virus, retrovirus, lentivirus, and adeno-associated virus. Preferably the vector is a plasmid. In some aspects of the invention, a vector is capable of replication in the cell to which it is introduced; in other aspects the vector is not capable of replication. In some preferred aspects of the present invention, the vector is unable to mediate the integration of the vector sequences into the genomic DNA of a cell. An example of a vector that can mediate the integration of the vector sequences into the genomic DNA of a cell is a retroviral vector, in which the integrase mediates integration of the retroviral vector sequences. A vector may also contain transposon sequences that facilitate integration of the coding region into the genomic DNA of a host cell.

Selection of a vector depends upon a variety of desired characteristics in the resulting construct, such as a selection marker, vector replication rate, and the like. An expression vector optionally includes expression control sequences operably linked to the coding sequence such that the coding region is expressed in the cell. The invention is not limited by the use of any particular promoter, and a wide variety is known. Promoters act as regulatory signals that bind RNA polymerase in a cell to initiate transcription of a downstream (3′ direction) operably linked coding sequence. The promoter used in the invention can be a constitutive or an inducible promoter. It can be, but need not be, heterologous with respect to the cell to which it is introduced.

Another option for increasing the expression of a gene like OPAL1 or one or more gene products as described in Table 1 wherein higher expression levels are predictive for favorable outcome is to reduce the amount of methylation of the gene. Demethylation agents, therefore, can be used to re-activate the expression of OPAL 1 or other gene product in cases where methylation of the gene is responsible for reduced gene expression in the patient.

For other genes identified herein as being correlated with therapeutic failure or without outcome in ALL, such as infant leukemia or pediatric ALL, especially B precursor ALL, especially pediatric B precursor ALL, high expression of the gene is associated with a negative outcome rather than a positive outcome. In the present invention, these genes/gene products (see Table 1) are selected from the group consisting of LGALS1/galectin, DOK1, GST1, CCL5/RANTES, PRG1, CD44, ATP2C1, SPARC—at least three genes/gene products from this list other than DOK1 and ATP2C1, preferably at least four other than DOK1 and ATP2C1, at least 5 from this list, at least 6 from this list, at least 7 from this list, and at least all 8 genes/gene products from this list. In such instances, where the expression levels of these eight genes are high, the predicted therapeutic outcome in such patients is therapeutic failure for traditional therapies. In such case, more aggressive approaches to traditional therapies and/or experimental therapies may be attempted.

The eight genes described above (negative outcome) accordingly represent novel therapeutic targets, and the invention provides a therapeutic method for reducing (inhibiting) the amount and/or activity of these polypeptides of interest in a leukemia patient. Preferably the amount or activity of the selected gene product is reduced to less than about 90%, more preferably less than about 75%, most preferably less than about 25% of the gene expression level observed in the patient prior to treatment.

A cell manufactures proteins by first transcribing the DNA of a gene for that protein to produce RNA (transcription). In eukaryotes, this transcript is an unprocessed RNA called precursor RNA that is subsequently processed (e.g. by the removal of introns, splicing, and the like) into messenger RNA (mRNA) and finally translated by ribosomes into the desired protein. This process may be interfered with or inhibited at any point, for example, during transcription, during RNA processing, or during translation. Reduced expression of the gene(s) leads to a decrease or reduction in the activity of the gene product and, in cases where high expression leads to a theapeuric failure, an expected therapeutic success.

The therapeutic method for inhibiting the activity of a gene whose high expression (table 1) is correlated with negative outcome/therapeutic failure involves the administration of a therapeutic agent to the patient to inhibit the expression of the gene. The therapeutic agent can be a nucleic acid, such as an antisense RNA or DNA, or a catalytic nucleic acid such as a ribozyme, that reduces activity of the gene product of interest by directly binding to a portion of the gene encoding the enzyme (for example, at the coding region, at a regulatory element, or the like) or an RNA transcript of the gene (for example, a precursor RNA or mRNA, at the coding region or at 5′ or 3′ untranslated regions) (see, e.g., Golub et al., U.S. patent application Publication No. 2003/0134300, published Jul. 17, 2003). Alternatively, the nucleic acid therapeutic agent can encode a transcript that binds to an endogenous RNA or DNA; or encode an inhibitor of the activity of the polypeptide of interest. It is sufficient that the introduction of the nucleic acid into the cell of the patient is or can be accompanied by a reduction in the amount and/or the activity of the polypeptide of interest. An RNA captamer can also be used to inhibit gene expression. The therapeutic agent may also be protein inhibitor or antagonist, such as small non-peptide molecule such as a drug or a prodrug, a peptide, a peptidomimetic compound, an antibody, a protein or fusion protein, or the like that acts directly on the polypeptide of interest to reduce its activity.

The invention includes a pharmaceutical composition that includes an effective amount of a therapeutic agent as described herein as well as a pharmaceutically acceptable carrier. These therapeutic agents may be agents or inhibitors of selected genes (table 1). Therapeutic agents can be administered in any convenient manner including parenteral, subcutaneous, intravenous, intramuscular, intraperitoneal, intranasal, inhalation, transdermal, oral or buccal routes. The dosage administered will be dependent upon the nature of the agent; the age, health, and weight of the recipient; the kind of concurrent treatment, if any; frequency of treatment; and the effect desired. A therapeutic agent identified herein can be administered in combination with any other therapeutic agent(s) such as immunosuppressives, cytotoxic factors and/or cytokine to augment therapy, see Golub et al, Golub et al., U.S. patent application Publication No. 2003/0134300, published Jul. 17, 2003, for examples of suitable pharmaceutical formulations and methods, suitable dosages, treatment combinations and representative delivery vehicles.

The effect of a treatment regimen on an acute leukemia patient can be assessed by evaluating, before, during and/or after the treatment, the expression level of one or more genes as described herein. Preferably, the expression level of gene(s) associated with outcome, such as OPAL1 or other gene as described above (preferably, favorable outcome, but also, negative outcome), may be monitored over the course of the treatment period. Optionally gene expression profiles showing the expression levels of multiple selected genes associated with outcome can be produced at different times during the course of treatment and compared to each other and/or to an expression profile correlated with outcome.

Screening for Therapeutic Agents

The invention further provides methods for screening to identify agents that modulate expression levels of the genes identified herein that are correlated with outcome, risk assessment or classification, cytogenetics or the like. Candidate compounds can be identified by screening chemical libraries according to methods well known to the art of drug discovery and development (see Golub et al., U.S. patent application Publication No. 2003/0134300, published Jul. 17, 2003, for a detailed description of a wide variety of screening methods). The screening method of the invention is preferably carried out in cell culture, for example using leukemic cell lines (especially B-precursor ALL cell lines) that express known levels of the therapeutic target, such as OPAL1 or other gene product as otherwise described herein (see also Table 1). The cells are contacted with the candidate compound and changes in gene expression of one or more genes relative to a control culture or predetermined values based upon a control culture are measured. Alternatively, gene expression levels before and after contact with the candidate compound can be measured. Changes in gene expression (above or below a predetermined value) indicate that the compound may have therapeutic utility. Structural libraries can be surveyed computationally after identification of a lead drug to achieve rational drug design of even more effective compounds.

The invention further relates to compounds thus identified according to the screening methods of the invention. Such compounds can be used to treat infant leukemia and/or pediatric ALL, especially B precursor ALL as appropriate, and can be formulated for therapeutic use as described above.

Active analogs, as that term is used herein, include modified polypeptides. Modifications of polypeptides of the invention include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

In certain aspects of the present invention, a therapeutic method may rely on an antibody to one or more gene products predictive of outcome, preferably to one or more gene product which otherwise is predictive of a negative outcome, so that the antibody may function as an inhibitor of a gene product. Preferably the antibody is a human or humanized antibody, especially if it is to be used for therapeutic purposes. A human antibody is an antibody having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example. Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. U.S.A., 90:2551-2555 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boemer et al. are also available for the preparation of human monoclonal antibodies (Cole et al., Monoclonal Antibodies and Cancer Therapy, Alan R. Liss, p. 77 (1985); Boerner et al., J. Immunol., 147(l):86-95 (1991)).

Antibodies generated in non-human species can be “humanized” for administration in humans in order to reduce their antigenicity. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Residues from a complementary determining region (CDR) of a human recipient antibody are replaced by residues from a CDR of a non-human species (donor antibody) such as mouse, rat or rabbit having the desired specificity. Optionally, Fv framework residues of the human immunoglobulin are replaced by corresponding non-human residues. See Jones et al., Nature, 321:522-525 (1986); Riechmann et al., Nature, 332:323-327 (1988); and Presta, Curr. Op. Struct. Biol., 2:593-596 (1992). Methods for humanizing non-human antibodies are well known in the art. See Jones et al., Nature, 321:522-525 (1986); Riechmann et al., Nature, 332:323-327 (1988); Verhoeyen et al., Science, 239:1534-1536 (1988); and (U.S. Pat. No. 4,816,567).

Laboratory Applications

The present invention further includes an exemplary microchip for use in clinical settings for detecting gene expression levels of one or more genes described herein as being associated with outcome, risk classification, cytogenics or subtype in infant leukemia and pediatric ALL. In a preferred embodiment, the microchip contains DNA probes specific for the target gene(s). Also provided by the invention is a kit that includes means for measuring expression levels for the polypeptide product(s) of one or more such genes, including any of the genes listed in Table 1 below, preferably one or more of midkine (neurite growth-promoting factor 2), CHST 10 (carbohydrate sulfotransferase 1 or HNK1-sulfotransferase), PHYN (phytanoyl-CoA hydroxylase), IFI44L (Interferon-induced protein 44-like, C1orf29), OPAL 1, CDK8 (cyclin-dependent kinase 8), DOK1 (docking protein 1-62 kD and downstream of tyrosine kinase 1) and ATP2C1 (ATPase-Ca++ transporting type 2C member 1), as positive outcome predictor genes/gene products or one or more of LGALS1/galectin, DOK1, GST1, CCL5/RANTES, PRG1, CD44, ATP2C1, SPARC, as negative outcome predictors, preferably a combination of these genes/gene products. In certain preferred embodiments, the microchip contains DNA probes for all 26 genes which are set forth in Table 1 or any of the two sets of eight gene described above alone with at least one additional gene taken from those set forth in Table 1. In a preferred embodiment, the kit is an immunoreagent kit and contains one or more antibodies specific for the polypeptide(s) of interest.

Relevant portion of the below cited references are referenced and incorporated herein. In addition, previously published WO 2004/053074 (Jun. 24, 2004) is incorporated by reference in its entirety herein.

In the present invention, sophisticated computational tools and statistical methods were used to reduce our comprehensive molecular profiles to a more limited set of 26 genes (a gene expression “classifier”) that is highly predictive of overall outcome in our pediatric ALL cohort (p<0.0001) (Table 1; a more detailed data presentation is provided in Table 2C, hereinbelow). The 26 genes in our molecular classifier are highly interesting and include several signaling, adhesion, and growth regulatory proteins (RhoGEF4, FYB, HNK-1 sulfotransferase, SMAD1, type 3 inositol-1,4,5 triphosphate receptor, MONDOA, DOK1, CDK8, CD44, CCL5/RANTES, galectin, SPARC) and novel genes not previously known to have a role in hematopoiesis or leukemogenesis (DREBIN, MIDKINE, and the hypothetical protein FLJ20154 or OPAL1 which was recently cloned and characterized by our laboratory and is a novel WBP-1 related protein) (Table 1). Of these 26 genes, 18 were positively associated with outcome (high expression is predictive of CCR-continuous complete remission) and 8 were negatively associated with outcome (high expression predicts treatment failure) (Table 1). We then used stepwise logistic regression to build a “best model” for predicting outcome, using expression values from each of the 27 probe sets (26 genes) (Table I) and the 4 clinical covariates (WBC count, age, sex and t(12; 21)). To our surprise, the final model included 8 genes (9 Affy probe sets) and none of the clinical variables as outcome predictors. Thus, the present invention may measure all 26 gene products.

TABLE 1
A 26 Gene Expression Classifier for
Outcome Prediction in Pediatric ALL¹

| | | High | |
|---|---|---|---|
| | | Expres- | Gene Description and Representative |
| Rank | p-value | sion | Identifier/Accession Number |
| 1* | 0.000001 | CCR | MIDKINE (neurite growth-promoting |
| | | | factor 2) NM_002391; |
| | | | NM_001012333; NM_001012334 |
| 2* | 0.000072 | CCR | MIDKINE same as above |
| 3 | 0.000163 | CCR | ARHGEF (rho guanine nucleotide |
| | | | exchange factor 4) NM_015320; |
| | | | NM_032995 |
| 4 | 0.000324 | CCR | FYB (FYN-binding protein; FYB) |
| | | | NM_001465; NM_199335 |
| 5* | 0.000065 | CCR | CHST10 (carbohydrate sulfotransferase |
| | | | 10) NM_004854 |
| 6 | 0.000055 | CCR | DREBIN 1 NM_004395; NM_080881 |
| 7 | 0.000893 | CCR | SMAD1 NM_005900; NM_001003688 |
| 8 | 0.000266 | CCR | HABP4 (hyaluronan-binding protein) |
| | | | NM_014282 |
| 9* | 0.000390 | CCR | PHYN (phytanoyl-CoA hydroxylase; |
| | | | Refsum disease) NM_006214 |
| 10* | 0.000494 | CCR | IFI44L (interferon-induced protein 44- |
| | | | like) (C1orf29) NM_006820 |
| 11 | 0.000398 | CCR | Human cDNA FLJ30991 fis, clone |
| | | | HLUNG1000041 AK055553 |
| 12 | 0.000481 | CCR | ITPR3 (inositol 1,4,5-triphosphate |
| | | | receptor, type 3) NM_002224 |
| 13 | 0.000580 | CCR | NMT2 (N-myristoyltransferase 2) |
| | | | NM 004808 |
| 14* | 0.000579 | CCR | OPAL1; C10orf26 (hypothetical protein |
| | | | FLJ20154) See WO 2004/053074 |
| | | | (Jun. 24, 2004) |
| 15 | 0.001767 | CCR | NPY (neuropeptide Y) NM_000905; |
| | | | M15789; K01911; M84755 |
| 16 | 0.000999 | CCR | JAG1 (jagged 1; Alagille syndrome) |
| | | | NM_000214 |
| 17 | 0.000510 | CCR | MONDOA (Mix Interactor) NM_014938; |
| | | | AF312918 |
| 18 | 0.002519 | CCR | EFN-B2 (ephrin-B2) NM_004093; |
| | | | BC105955; BC105956; BC105957 |
| 19* | 0.000833 | CCR | CDK8 (cyclin-dependent kinase 8) |
| | | | NM_01260; BC105950; BC107601 |
| 1 | 0.000299 | Fail | LGALS1 (lectin, galactoside-binding, |
| | | | soluble, galectin 1) NM_002305; |
| 2* | 0.000160 | Fail | DOK1 (docking protein 1, 62 kD, |
| | | | downstream of tyrosine kinase 1) |
| | | | NM_001381 |
| 3 | 0.000111 | Fail | GSTθ1 (glutathione-S-transferase omega |
| | | | 1) NM_004832 |
| 4 | 0.000229 | Fail | CCL5 (chemokine ligand 5)/RANTES |
| | | | NM_002985 |
| 5 | 0.000687 | Fail | PRG1 (proteoglycan 1) NM_002727 |
| 6 | 0.000629 | Fail | CD44 |
| | | | NM_000610; AY101193; AY101192; |
| | | | NM_001001392; NM_001001391; |
| | | | NM_001001390; NM_001001389 |
| 7* | 0.000371 | Fail | ATP2C1 (ATPase, Ca++ transporting, |
| | | | type 2C, member 1) |
| | | | NM_014382; NM_001001487; |
| | | | NM_001001486; NM_001001485 |
| 8 | 0.000475 | Fail | SPARC (secreted protein, acidic, |
| | | | cysteine-rich; osteonectin) NM_003118 |

¹Footnotes to Table 1:
1. Note that these 27 probe sets are derived from 26 distinct genes; the two top ranked probe sets (Table 3) are derived from different regions of the same gene, MIDKINE. Note that Accession Numbers are representative of the gene in humans (including natural variants); other accession numbers may also be applicable (see WO 2004/053074 (Jun. 24, 2004), especially Table 42.
2. Column 1: Rank: The relative rank of the predictive gene.
3. Column 2: p value: The p value of the power of this gene to distinguish between CCR and treatment failure as an independent variable, determined by two-sided t-test.
4. Column 3: Whether higher levels of expression of the gene was associated with CCR (Continuous Complete Remission) or failure.
5. Column 4: Gene Description/Human Chromosome Location

These preliminary data, form the cornerstone of our Leukemia & Lymphoma Society Specialized Center of Research, focused on Acute Lymphoblastic Leukemia (ALL) (20). To fully exploit new technologies for the identification of novel genes and proteins that can be used to improve risk classification and to identify new targets for therapy in the clinic, and, in turn to understand the fundamental biology of these genes and proteins in the context of leukemic cells in the laboratory, we have built a highly interacfive multidisciplinary team of laboratory and computational scientists from the University of New Mexico, Sandia National Laboratories, the Fred Hutchinson Cancer Research Center, and the intramural proteomics program at the National Cancer Institute (the NCI/FDA Clinical Proteomics Program). The four projects that we have proposed are highly integrated and span from the initial use of leukemia patient samples to further develop, refine, and validate both gene expression and proteomic classifiers for outcome prediction (Projects 1 and 2) to more fundamental scientific studies focused on increasing our understanding of the function of these genes using hematopoietic and leukemic cell lines and animal models (Project 3) to biochemical, computational and modeling studies in which we move from “lists” of genes and proteins to understanding their interactions in the context of signaling pathways and networks (Projects 2 and 4). These fundamental studies should allow us in turn to further refine our outcome classifiers and gain insights for how these novel genes, proteins, and pathways may be exploited for new and hopefully more effective therapeutic interventions in ALL. Distinguishing our work to date and the Projects proposed in this SCOR are our very significant collaborations with scientists at Sandia National Laboratory, located only 15 minutes from UNM in Albuquerque. Sandia National Laboratory (www.sandia.gov), a DOE laboratory managed by Lockheed Martin, has 8,600 employees (>5000 PhDs) in Albuquerque and also manages Sandia-Livermore Laboratory in California. Through our extensive collaborations with Sandia scientists, our SCOR team brings unparalleled capabilities in computational biology, massively parallel computing, novel cellular imaging techniques, the development of novel molecular probes and functionalized nanoprobes, and mathematical algorithms and software for the dynamic modeling of cell regulatory pathways and signaling networks in normal and leukemic cells. These sophisticated technologies and collaborative arrangements are particularly apparent.

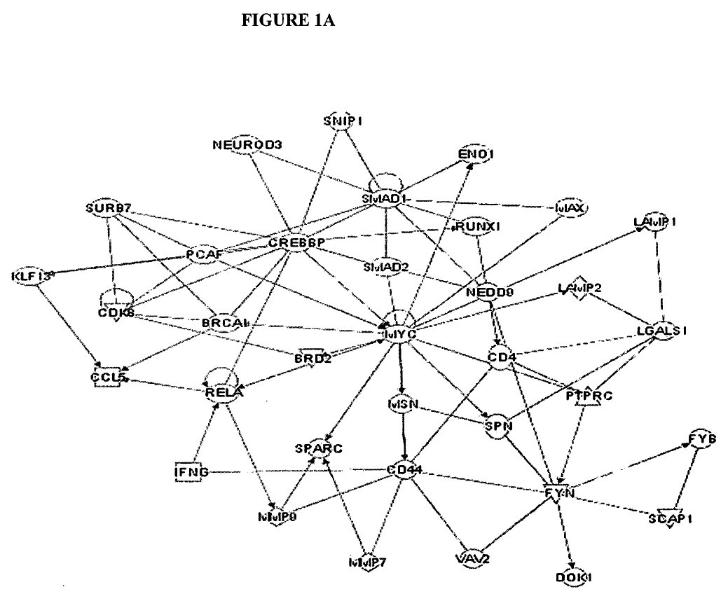

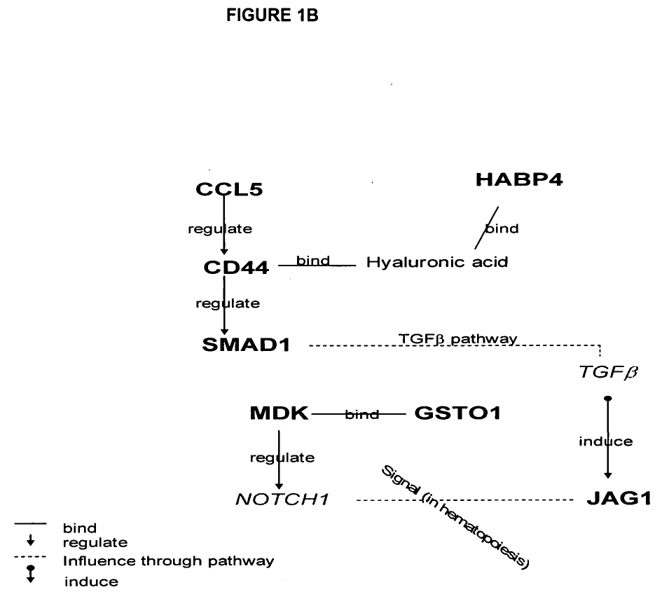

One of the primary overall goals of our LLS SCOR is to begin to transition from “lists” of outcome genes and proteins to an understanding of the relationships between these genes (and proteins), if one exists, and their functional roles in cell regulatory and signaling networks. We are particularly interested in determining how these pathways are perturbed in leukemic cells. Toward this end, we have begun to use computational tools such as Ingenuity Pathways Analysis (www.ingenuity.com) and other tools developed with our collaborators at Sandia National Laboratory (see Project 4). Interestingly, 8 (CCL5/RANTES, CD44, CDK8, DOK1, FYB, LGALS1, SMAD1, SPARC) of the 26 genes (see Table 1, below) identified in our initial gene expression classifier are linked in a cell death regulatory network (FIG. 1, below, left). Seven genes (CCL5/RANTES, CD44, SMAD1, MDK, GSTθ1, JAG1, and HABP4) are also components of a chemokine/CD44 signaling pathway that will be investigated in detail (FIG. 1 below, right). Finally, two genes (ITPR3 and ATP2C1) that are critical regulators of intracellular calcium ion transport and apoptosis will by dynamically modeled in Project 4. Continued analysis of these pathways, as proposed in this LLS SCOR, may provide new insights into how these genes promote leukemogenesis and effect therapeutic responsiveness.

Significant advances in the treatment of pediatric ALL have been achieved through the use of risk classification schemes that target children to increasing therapeutic intensities based on their relapse risk. However, current classification schemes do not fully reflect the molecular heterogeneity of the disease and do not precisely identify those children more prone to relapse or those who could be cured with less intensive regimens. To improve risk classification and outcome prediction in ALL, gene expression profiles were obtained using oligonucleotide arrays in a retrospective case control study of 220 children with B precursor ALL, balanced for outcome (continuous complete remission (CCR) vs. failure at 4 years) across several established prognostic variables (age, sex, WBC, karyotype). Using multiple statistical methods and computational tools, these comprehensive gene expression profiles were reduced to a 26 gene expression classifier that was highly predictive of overall outcome (two tailed p values ranging from 0.00001-0.001). Each of these 26 genes was shown to provide additional prognostic information relative to established prognostic variables (p<0.01). The 26 genes include signaling, adhesion, and growth regulatory proteins (RhoGEF4, FYB, HNK-1 sulfotransferase, SMAD1, HABP4, PHYN, IF144L, JAG1, EFN-B2, type 3 inositol-1,4,5 triphosphate receptor, MONDOA, DOK1, CDK8, CD44, CCL5/RANTES, galectin, SPARC) and novel genes not previously known to play a role in hematopoiesis or leukemogenesis (DREBIN, MIDKINE, and the hypothetical protein FLJ20154 or OPAL1 which have cloned and characterized). High expression of 18 of the 26 genes was predictive of CCR while high expression of the remaining 8 genes (LGALS1/galectin, DOK1, GST1, CCL5/RANTES, PRG1, CD44, ATP2C1, SPARC) was predictive of treatment failure. Interestingly, 8 of the 26 genes are linked in a cell death regulatory network; 7 genes are components of a chemokine/CD44 signaling pathway; and 2 genes are critical regulators of intracellular calcium ion transport and apoptosis. Using stepwise logistic regression on the expression values of the 26 genes and 4 established prognostic variables (sex, age, WBC, t(12;21)), the best predictive outcome model was built using 9 genes alone (MIDKINE, CHST10, PHYH, IF144L, OPAL1, CDK8, DOK1, ATP2C1, SPARC). This 9 gene predictive model was then tested for its ability to predict outcome in two independent B precursor ALL cohorts: 1) a series of 198 B precursor ALL cases previously published by Yeoh et al. (Cancer Cell 2002 1:133) where our 9 gene model was found to predict outcome with high statistical significance (p<1.0-8); and, 2) a series of 59 B precursor ALL patients treated with a distinct modified BFM regimen CCG-1961 (p=0.002; W. L. Carroll et al, in preparation). The results of the present invention demonstrate that gene expression profiling can yield unique genes and classifiers that can improve outcome prediction and risk classification in ALL. These studies provide new insights into how these genes and pathways may promote leukemogenesis and effect therapeutic responsiveness.

Development and Use of Gene Expression Classifiers for Improved Outcome Prediction in ALL

The major challenge in pediatric acute lymphoblastic leukemia (ALL) is to improve risk classification schemes and outcome prediction in order to: 1) identify those children who are most likely to relapse who require intensive or novel regimens for cure; and 2) identify those children who can be cured with less intensive regimens with fewer toxicities and long term side effects. In contrast to pediatric ALL, overall outcome in adult ALL remains poor and risk classification schemes are rarely employed. To identify novel genes predictive of outcome in ALL that could be used to improve risk classification, we used oligonucleotide arrays to obtain comprehensive gene expression profiles in a retrospective case control study of 254 children with ALL. Using computational and statistical methods, we developed a 26 gene classifier that was highly predictive of outcome; in a step wise logistic regression analysis, a subset of 9 of these genes highly predictive of outcome compared to traditional prognostic variables (p<0.0001). Prior to its implementation in the context of clinical trials, it is essential to determine if this classifier is reproducible in an independent prospective cohort of cases. Many of the genes in this classifier encode signaling and adhesion proteins, some of which were not previously known to play a role in hematopoiesis or leukemogenesis, others of which are novel. Thus, further insights into the functional role of these genes in normal and leukemic cells may provide new insights for leukemogenesis and new pathways that can be exploited for the future development of more effective therapies. The specific aims are to:
1. Extend and prospectively validate a gene expression classifier for outcome prediction in pediatric ALL in two statistically-designed prospective cohorts of pediatric ALL patients registered to NCI-sponsored clinical trials. New high density expression arrays (Affymetrix HG_U133 Plus2.0 Arrays containing 54,000 probe sets) will first be used to obtain comprehensive expression profiles in a new, independent, statistically-designed, prospective cohort of 150 uniformly treated pediatric ALL patients registered to an NCI-sponsored trial conducted by the Children's Oncology Group (GOG). Sophisticated computational and statistical tools will be used to derive a refined gene expression classifier which will be compared and contrasted with our initial classifier. Using automated quantitative RT-PCR, the predictive power of the genes in the molecular classifier will then be tested prospectively in a second statistically-designed independent validation cohort of 544 uniformly treated children with ALL, in whom all clinical and prognostic factors, including minimal residual disease at end induction, have been collected and established.
2. Test the clinical utility of the molecular classifier and predictive genes developed in pediatric ALL patients for outcome prediction in adult ALL patients (in collaboration with Jerry Radich of the FHCRC, Project I Co-Investigator). The predictive power of the molecular classifier will be tested using automated quantitative RT-PCR in a statistically designed prospective cohort of 150 adult ALL patients registered to NCI-sponsored treatment trials conducted by the Southwest Oncology Group (SWOG). If the classifier developed in pediatric patients fails to predict outcome in adult ALL, then a separate molecular classifier will be developed following comprehensive molecular profiling of this cohort using Affymetrix HG_U133 Plus2. 0 Arrays).
3. Correlate gene expression profiles with proteomic profiles derived from a linearly dynamic reverse phase protein microarray system (in collaboration with the NCI/FDA Clinical Proteomics Program in Project 2), using paired samples from the same patient cohorts.
4. Develop an understanding of the functional relationships of these novel genes and proteins in adhesion and signaling networks and regulatory pathways in normal blood cells, and, determine how these networks are perturbed in leukemic cells using cell lines, animal models, and sophisticated imaging and computational tools for the dynamic modeling of signaling pathways (in collaboration with Projects 2, 3, and 4).
   Scientific Background and Clinical Significance of the Proposed Work

While remarkable advances have been made in the treatment of ALL, significant challenges remain. Although the use of modern combination chemotherapy and post-induction therapeutic intensification now yield long-term remissions in up to 75% of children with ALL, 25-30% of children will ultimately relapse with highly refractory disease (1-4). Conversely, in order to achieve such high remission rates, up to one-third of children with ALL are likely over-treated and may well be cured using less intensive regimens resulting in fewer acute toxicities and long term side effects. Thus, the major challenge in pediatric ALL is to improve and refine risk classification schemes in order to precisely tailor therapeutic approaches in individual patients. The vast majority of pediatric ALL cases (>85%) are of the B precursor cell phenotype (Table 2, below). In order to modulate the intensity of therapy relative to a patient's risk of relapse, B precursor ALL patients are currently stratified into “low”, “standard,” “high,” and “very high” risk groups using laboratory and clinical parameters: patient age, sex, white blood cell count (WBC) at disease presentation, and the presence or absence of specific cytogenetic abnormalities (Table 1). The frequently recurring genetic abnormalities that help define these risk groups include: t(12;21) [TEL-AML1], t(1;19) [E2A-PBX], t(4;11) [AF4-MLL], t(9;22) [BCR-ABL], hyperdiploidy (or trisomy of chromosomes 4, 10, and 17), and hypodiploidy. The rate of disappearance of leukemic cells during induction (assessed morphologically or by other quantitative measures of residual disease) has also been established as an important assessment of early therapeutic response and as a means of targeting children for therapeutic intensification (4-8). Yet despite these efforts, risk classification for pediatric ALL remains imprecise. We still cannot prospectively identify the 10% of low risk, 20% of standard risk, 30% of high risk, and 70% of children with very high risk disease who will relapse (Table 2B). The majority of children with ALL, nearly 60%, fall into the low and standard risk groups, which remain highly heterogeneous in clinical and cytogenetic features. Within these lower risk groups, it is estimated that we are over-treating at least one third of patients who experience significant toxicities and short and long term side effects in multiple organ systems. Thus, while advanced when compared to many other cancers, current risk classifications schemes do not reflect the extensive molecular heterogeneity of pediatric ALL and do not adequately target children for therapy. In contrast to pediatric ALL, outcome in adult ALL remains dismal, with overall survival less than 40%. This poor outcome is not solely explained by an increased frequency of poor risk cytogenetic abnormalities such as the t(9;22) in adults (22%) vs. children (3%). Although adult Ph+ ALL patients have been targeted to regimens incorporating imatinib/Gleevec (the BCR-ABL tyrosine kinase inhibitor), there are no risk classification schemes for the majority of patients.

TABLE 2B
Risk Classification Scheme for Pediatric ALL (Children's Oncology Group)

| | | % of Pediatric |
|---|---|---|
| Risk Category | Clinical, Laboratory, and Cytogenetic Parameters | ALL |
| B Precursor ALL: | NCI standard risk: (age 1-10 yrs and WBC < 50,000/μl), and, | 25% |
| Low Risk | Low risk cytogenetic abnormalities: t(12; 21) or hyperdiploid DNA | |
| 91% 4 Yr Event Free | content (trisomies of chromosomes 4, 10, and/or 17), and, | |
| Survival (EFS) | Rapid clearance of marrow blasts during induction chemotherapy, | |
| | and, | |
| | No extramedullary disease | |
| B Precursor ALL: | NCI standard risk group without low risk or high risk cytogenetic | 35% |
| Standard Risk | abnormalities, or, | |
| 82% 4 Yr EFS | Children with low risk cytogenetic abnormalities who are either | |
| | NCI high risk (age > 10 Yrs or WBC > 50,000/μl) or who have | |
| | slow clearance of marrow blasts during induction | |
| B Precursor ALL: | NCI high risk group (age > 10 Yrs or WBC > 50,000/μl), or, | 20% |
| High Risk | Intermediate to high risk cytogenetic abnormalities: 11q23/MLL | |
| 72% 4 Yr EFS | rearrangements, particularly t(4; 11) or t(1; 19) | |
| B Precursor ALL: | High risk cytogenetic abnormalities: t(9; 22) or hypodiploid DNA | 4-5% |
| Very High Risk | content (<45 chromosomes or DNA Index < 0.81) | |
| 33% 4 Yr EFS | Induction therapy failure | |
| B-ALL | Specific cytogenetic abnormalities: t(8; 14) and variants | 1-2% |
| T-ALL | Defined by immunophenotype; no current risk classification | 13% |
| | employed | |

Previous Studies/Preliminary Data

As striking differences in therapeutic response may be observed in ALL patients with the same cytogenetic abnormalities or within the same risk classification group, it is likely that other genetic abnormalities and functional disruption of critical cellular pathways in leukemic cells also impact disease biology and response. Thus, we and others have hypothesized that large scale genomic technologies that measure gene expression and proteomic profiles in leukemic cells will yield new genes for improved risk classification and outcome prediction, and new insights for leukemogenesis. Funded under the NCI Director's Challenge Program: Toward a Molecular Classification of Tumors (2000-2005), we worked with two NCI Cooperative Oncology Groups (COG and SWOG) to design retrospective patient cohorts from which we derived rigorously cross-validated gene expression profiles in children and adults with acute leukemia. During this period, we built a highly collaborative multidisciplinary laboratory, statistical, and computational team; developed reproducible and sensitive methods for performing gene expression arrays; designed data warehouses for storage of large gene expression datasets fully annotated with clinical, outcome, and experimental information; and developed and applied robust statistical and computational methods and novel visualization tools for array data analysis. The results of these analyses are now published, in press, or submitted for publication (15-34) and our fully annotated gene expression and clinical datasets are publicly available at the NCI Gene Expression Data Portal website (http://gedp.nci.nih.gov/dc).

One of our most interesting and potentially important datasets was obtained by gene expression profiling (using Affymetrix HG_U95Av2 oligonucleotide arrays containing 12,625 probe sets) of the diagnostic (pre-treatment) leukemic samples from a retrospective cohort of 254 children with ALL. These 254 patients were selected in the statistical design of a series of retrospective case-control studies (focused on race, gender, induction response, and ALL-associated cytogenetic abnormalities) where cases (failure within 4 years) were balanced with controls (continuous complete remission (CCR) at 4 or more years). The statistical design of this retrospective case control study proved to be crucial for the successful computational modeling of outcome genes. In general, to increase the likelihood that supervised learning methods applied to gene expression datasets will be successful in identify genes significantly associated with outcome, it is critical to balance the cases under analysis 50:50 (CCR: failure). If a case series is imbalanced relative to outcome, then supervised learning methods generally fail to yield significant genes, or, genes derived cannot be validated in independent test sets. In pediatric ALL, up to 75% of patients attain CCR; thus we had to deliberately “over-sample” for cases with treatment failure. The 254 patients included a series of 220 B precursor ALL and a small case control study of 34 T-ALL patients. The complete clinical and outcome data on these 254 patients, as well as the gene expression data, can be found at the NCI Gene Expression Data Portal website (http://gedp.nci.nih.gov/dc; Experiment Accession ID: 754). Although we have successfully modeled outcome genes in T-ALL, the remainder of this discussion will focus on the results obtained in the 220 B precursor ALL patients. Although our B precursor ALL cases were highly selected, their clinical and outcome characteristics were similar to those in the general pediatric ALL population: in our cohort, CCR was associated with a lower WBC (p<0.006), younger age (p<0.01), female sex (p=0.04), and the presence of the t(12;21) (p<0.002). There were two few cases with t(9;22), t(1;19), or t(4;11) to be able to successfully model outcome in these rarer cytogenetic groups.

Working with the computational biology and statistical team in Core B, multiple supervised learning algorithms (Bayesian Network Classification (11), Support Vector Machines (12)) and univariate statistical tools (Analysis of Variance (ANOVA) (13), TNoM (14), and Significance Analysis of Microarrays or SAM (15)) were used to identify genes significantly associated with outcome. Strikingly, although their relative rank differed, the same genes were repeatedly found using different computational approaches. The set of predictive genes identified using SAM, which forms our 26 gene classifier, is provided in Table 2. Of the 12,625 probe sets on the U95Av2 array, 8942 were expressed in at least one of the 220 ALL cases; from these 8942 sequences, SAM identified 27 probe sets that were highly associated with outcome. Note that these 27 probe sets are derived from 26 distinct genes; the two top ranked probe sets (Table 3) are derived from different regions of the same gene, MIDKINE. Of these 26 genes, 18 were positively associated with outcome (high expression is predictive of CCR) and 8 were negatively associated with outcome (high expression predicts treatment failure). A brief explanation of the data in Table 3 is provided below:
Column 1: Rank: The relative rank of the predictive gene.
Column 2: p value: The p value of the power of this gene to distinguish between CCR and treatment failure as an independent variable, determined by t-test.
Column 3: Whether higher levels of expression of the gene was associated with CCR or failure.
Columns 4/5: We further assessed the significance of these 27 sequences (26 genes) by examining whether each gene individually has prognostic value beyond that contained in clinical covariates: the 4 clinical characteristics (WBC count, age, sex and t(12;21) status) that we knew were significantly associated with outcome (p-values<0.05) in our cohort). We fit logistic regression models with the 4 clinical covariates and each one of the 27 genes as predictors of outcome. Table 3 gives p-values for likelihood ratio tests of the significance of each gene individually (column 5) and the collective effect of the clinical covariates (column 4). Each gene has significant predictive ability (p-value<0.001) beyond that contained in the clinical covariates; with several of the genes treated as an independent prognostic factor, the clinical covariates collectively retain significant predictive ability as well.
Column 6: Affymetrix oligonucleotide probe set identifier (U95Av2 array).
Columns 7/8: Gene Description/Human Chromosome Location

A Model Classifier for Predicting Outcome in B precursor ALL.

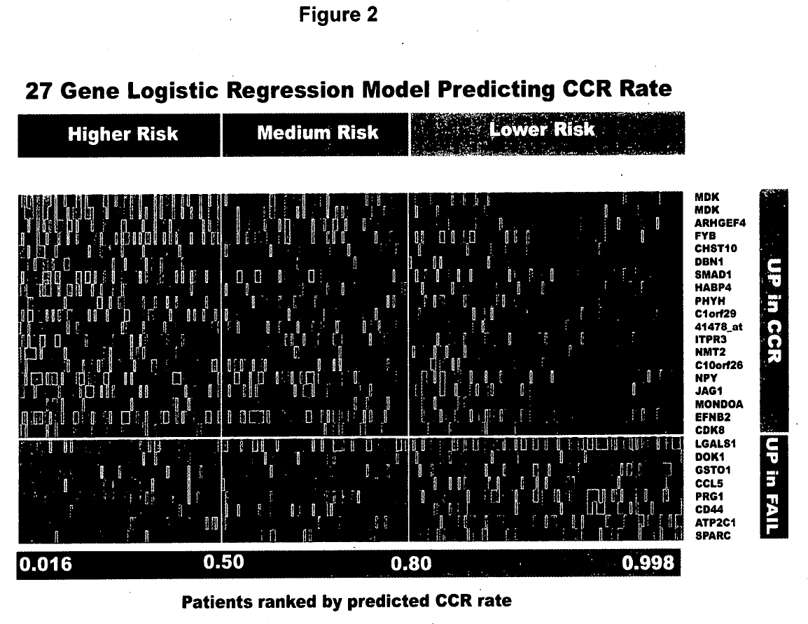

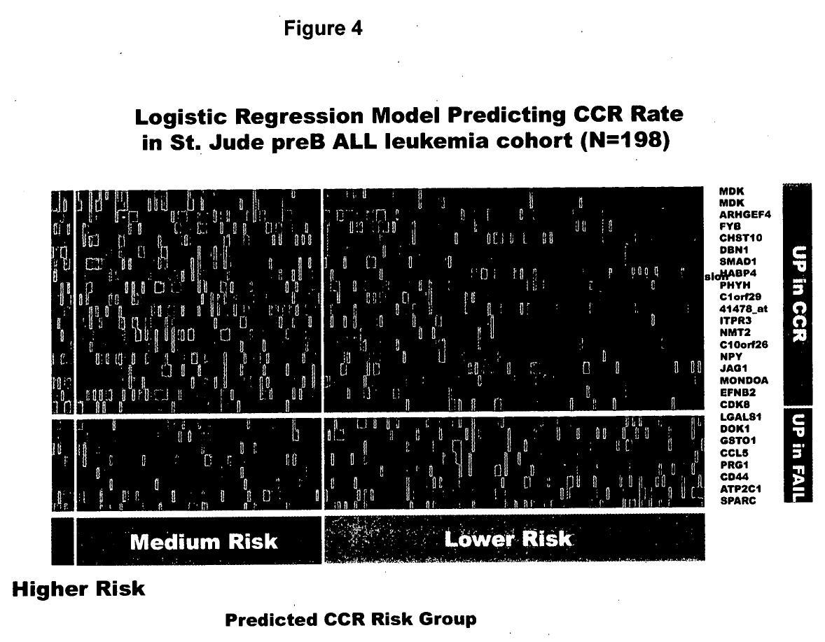

Stepwise logistic regression was utilized in SAS to build a “best model” for predicting outcome, using expression values from each of the 27 probesets (26 genes) (Table 2C) and the 4 clinical covariates (WBC count, age, sex and t(12; 21)). Bayes' theorem was used to adjust the final model to account for the over-sampling of patients that failed in our study. We first fit a model with all 31 variables and we found that about two-thirds of the variables no longer retained statistical significance (p-values>0.05) in the presence of all of the effects (all 31 variables). To identify a best subset of predictors, we performed stepwise logistic regression, using a significance level of 0.20 for predictors to enter the model and 0.05 for predictors to stay in the model. To our surprise, the final model included 8 genes (9 Affy probe sets) and none of the clinical variables as predictors of outcome. A backward elimination also led to the conclusion that none of the clinical predictors were significant in the presence of the gene expression values for these nine probe sets. These nine probe sets (8 genes), noted in Table 3, columns 1 and 6 by the (*), include: MIDKINE (both probe sets), CHST10, PHYN, IFI44L (C1orf29), OPAL1, CDK8, DOK1, and ATP2C1. Using this model, we developed a “score” of the likelihood of CCR, allowing us to place individual patients into different risk groups: a high risk group with poorer outcome (with scores<0.50), a medium risk group, (0.50≦score≦0.80), or low risk group with very good outcome (score>0.80). FIG. 1 shows the distribution of scores in our cohort of 220 B precursor ALL patients using all 26 genes in the expression classifier (FIG. 1) and or the 9 probe set classifier (FIG. 2). To further validate this model, we applied it to a previously published independent data set of ALL patients from St. Jude Children's Research Hospital (16). After removing the T-ALL patients and those with unknown outcome, 198 patients (175 CCR and 23 Failed) remained in this dataset. Using our model, we were able to predict outcome in this independent ALL case series with extremely high statistical significance (p-value<1.0⁻⁸). The distribution of outcome scores and expression of the predictive genes in this independent cohort of pediatric ALL cases are shown in FIG. 4.

The 26 genes in our molecular classifier are highly interesting and include several signaling, adhesion, and growth regulatory proteins (RhoGEF4, FYB, HNK-1 sulfotransferase, SMAD1, type 3 inositol-1,4,5 triphosphate receptor, MONDOA, DOK1, CDK8, CD44, CCL5/RANTES, galectin, SPARC) and novel genes not previously known to have a role in hematopoiesis or leukemogenesis (DREBIN, MIDKINE, and other uncharacterized open reading frames). Interestingly, both DREBIN and MIDKINE have been best characterized in the context of neural development and in neural stem cells; MIDKINE is a retinoic acid-responsive growth factor that is a ligand for the anaplastic lymphoma kinase ALK and α4β1 and α6β1 integrins (17). Interestingly, 8 (CCL5/RANTES, CD44, CDK8, DOK1, FYB, LGALS1, SMAD1, SPARC) of the 26 genes (Table 2) are linked in a cell death regulatory network. Seven genes (CCL5/RANTES, CD44, SMAD 1, MDK, GSTθ1, JAG 1, and HABP4) are also components of a chemokine/CD44 signaling pathway that will be investigated in detail in Two genes (ITPR3 and ATP2C1) are critical regulators of intracellular calcium ion transport and apoptosis.

TABLE 2C
A 26 Gene Expression Classifier for Outcome Prediction in Pediatric ALL

| | | | Likelihood Ratio Test | | | | |
|---|---|---|---|---|---|---|---|
| | T-test | | (p-values) | | | | |
| | | High | Significance | | Affy | | Human |
| | | Expres- | of clinical | Significance | Probe Set | | Chromosome |
| Rank | p-value | sion | variables | of gene | ID | Gene Description | Location |
| 1* | 0.000001 | CCR | 0.001333 | 0.007815 | 577_at | MIDKINE (neurite | 11p11.2 |
| | | | | | | growth-promoting | |
| | | | | | | factor 2) | |
| 2* | 0.000072 | CCR | 0.031001 | 0.004570 | 38124_at | MIDKINE | 11p11.2 |
| 3 | 0.000163 | CCR | 0.056628 | 0.000914 | 36524_at | ARHGEF (rho | 2q22 |
| | | | | | | guanine nucleotide | |
| | | | | | | exchange factor 4) | |
| 4 | 0.000324 | CCR | 0.022769 | 0.001030 | 41819_at | FYB (FYN-binding | 5p13.1 |
| | | | | | | protein; FYB) | |
| 5* | 0.000065 | CCR | 0.000704 | 0.000075 | 32058_at | CHST10 | 2q12.1 |
| | | | | | | (carbohydrate | |
| | | | | | | sulfotransferase 10) | |
| 6 | 0.000055 | CCR | 0.002805 | 0.000545 | 37981_at | DREBIN 1 | 5q35.5 |
| 7 | 0.000893 | CCR | 0.076917 | 0.001853 | 37280_at | SMAD1 | 4q31 |
| 8 | 0.000266 | CCR | 0.007022 | 0.000133 | 32970_at | HABP4 (hyaluronan- | 9q22-q31 |
| | | | | | | binding protein) | |
| 9* | 0.000390 | CCR | 0.024854 | 0.000643 | 32724_at* | PHYN (phytanoyl- | 10pter-p11 |
| | | | | | | CoA hydroxylase; | |
| | | | | | | Refsum disease) | |
| 10* | 0.000494 | CCR | 0.002723 | 0.000025 | 36927_at* | IFI44L (interferon- | 1p31.1 |
| | | | | | | induced protein 44- | |
| | | | | | | like) (C1orf29) | |
| 11 | 0.000398 | CCR | 0.015966 | 0.000277 | 41478_at | Human cDNA | |
| | | | | | | FLJ30991 fis, clone | |
| | | | | | | HLUNG1000041 | |
| 12 | 0.000481 | CCR | 0.021445 | 0.000507 | 37343_at | ITPR3 (inositol 1,4,5- | 6p21 |
| | | | | | | triphosphate receptor, | |
| | | | | | | type 3) | |
| 13 | 0.000580 | CCR | 0.155477 | 0.000828 | 41656_at | NMT2 (N- | 10p13 |
| | | | | | | myristoyltransferase | |
| | | | | | | 2) | |
| 14* | 0.000579 | CCR | 0.016025 | 0.000381 | 38652_at* | OPAL1; C10orf26 | 10q24.33 |
| | | | | | | (hypothetical protein | |
| | | | | | | FLJ20154) | |
| 15 | 0.001767 | CCR | 0.139056 | 0.000796 | 38604_at | NPY (neuropeptide Y) | 7p15.1 |
| 16 | 0.000999 | CCR | 0.235267 | 0.000977 | 35414_s_at | JAG1 (jagged 1; | 20p12.1 |
| | | | | | | Alagille syndrome) | |
| 17 | 0.000510 | CCR | 0.019658 | 0.001182 | 35260_at | MONDOA (Mlx | 12q21.3 |
| | | | | | | Interactor) | |
| 18 | 0.002519 | CCR | 0.242510 | 0.000909 | 34335_at | EFN-B2 (ephrin-B2) | 13q33 |
| 19* | 0.000833 | CCR | 0.002459 | 0.000060 | 1189_at* | CDK8 (cyclin- | 12q12 |
| | | | | | | dependent kinase 8) | |
| 1 | 0.000299 | Fail | 0.038092 | 0.001448 | 33412_at | LGALS1 (lectin, | 22q13.1 |
| | | | | | | galactoside-binding, | |
| | | | | | | soluble, galectin 1) | |
| 2* | 0.000160 | Fail | 0.013063 | 0.001759 | 34433_at* | DOK1 (docking | 2p13 |
| | | | | | | protein 1, 62 kD, | |
| | | | | | | downstream of | |
| | | | | | | tyrosine kinase 1) | |
| 3 | 0.000111 | Fail | 0.031630 | 0.000928 | 824_at | GSTθ1 (glutathione- | 10q25.1 |
| | | | | | | S-transferase omega 1) | |
| 4 | 0.000229 | Fail | 0.006548 | 0.000147 | 1403_s_at | CCL5 (chemokine | 17q11.2 |
| | | | | | | ligand 5)/RANTES | |
| 5 | 0.000687 | Fail | 0.053242 | 0.000522 | 32227_at | PRG1 (proteoglycan 1) | 10q22.1 |
| 6 | 0.000629 | Fail | 0.092880 | 0.000647 | 1126_s_at | CD44 | 11p13 |
| 7* | 0.000371 | Fail | 0.007099 | 0.000136 | 38684_at* | ATP2C1 (ATPase, | 3q22.1 |
| | | | | | | Ca++ transporting, | |
| | | | | | | type 2C, member 1) | |
| 8 | 0.000475 | Fail | 0.008262 | 0.000098 | 671_at* | SPARC (secreted | 5q31.3-32 |
| | | | | | | protein, acidic, | |
| | | | | | | cysteine-rich; | |
| | | | | | | osteonectin) | |

One of the most powerful predictors in the classifier, first identified using Bayesian network classification (11), was a novel sequence (Affymetrix 38652_at; NM_hypothetical protein FLJ20154) (Table 2, gene 14). Given the significance of this novel sequence, we proceeded to map, clone, and characterize this gene, which we named Outcome Predictor in Acute Leukemia 1 (OPAL1); this new gene is a novel member of the WBP1 family (see Project 3 for more discussion). The full length sequence of OPAL1 has been deposited in Genbank (accession number AY656171). As presented in the ASH Plenary Session (Abtract 1; Blood 102 (11): 4a, 2003), low OPAL1 was associated with induction failure (p=0.0036) while high OPAL1 was associated with event free survival (p=0.02), particularly in males (p=0.0004). While the 44% of the patients in our cohort achieved CCR, 86% of the patients with high OPAL1 expression achieved CCR. High levels of OPAL1 were associated with higher rates of CCR in children with a normal karyotype, in children with poor risk age (>10 years), and in children with WBC greater than 50,000 (normally considered poor risk) (27). In the independent set of cases from St. Jude, OPAL1 was predictive of outcome overall (p=0.006) and within their cohort of patients with t(12;21) (p=0.005). In the St. Jude series, all patients with t(12;21) and high OPAL1 achieved CCR (54/54), while less than 50% of the patients with t(12;21) and with low OPAL1 (3/7) achieved CCR. These results suggest that high levels of OPAL 1 may be useful in identifying those children with a particularly good outcome who may be cured using less intensive regimens.

### EXAMPLES

### Research Methods and Resources and Environment

All laboratory and computational methods for determining outcome determinative gene product lists may be found at http://hsc.unm.edu/crtc/WillmanResearch/ in PCT application WO WO 2004/053074 (Jun. 24, 2004), and related literature cited herein. Affymetrix GeneChip microarray analysis systems and a LIMS data server, as well as excellent robotic capabilities are used for propagation of clones from multiple genomes. Application of automated quantitative RT-PCR assays, are also used for validation studies. Related bioinformatics, biocomputing, mathematics and statistics are also used.

To refine and validate a gene expression classifier and identify top predictive genes for improved risk classification, outcome prediction, and therapeutic targeting in pediatric ALL.

Refinement of Gene Expression Classifiers in New Prospective Cohorts. Using Affymetrix HG_U133 Plus2.0 Arrays (containing 54,000 probe sets), we refine gene expression classifier by obtaining expression profiles in a new statistically designed, prospective case control study of 150 uniformly treated pediatric ALL patients registered to the COG/POG AlinC 17 treatment trials and the 9900 Risk Classification Study. Cases (failure within 4 years) are balanced with controls (CCR at 4 years). Genes predictive of outcome are identified using statistical methods and multiple supervised machine learning algorithms with full cross validation. These cases have been carefully selected and the experiment statistically designed and powered in collaboration with Dr. Harland Sather, the COG biostatistician who works closely with our team. First, we balanced ALL cases with CCR vs. failure in order to optimize the likelihood of success in identifying outcome genes using supervised machine learning algorithms; thus the patients in our study were highly selected and over-sampled for treatment failure relative to the general pediatric ALL population. Although we validated our initial results in an independent set of cases (16), we will further determine how translatable our results are to a new prospective set of cases more reflective of the general ALL population. Secondly, even though we studied 254 patient samples, some of the individual case-control studies encompassed within our overall design were relatively small (20-40 patients). In light of the critical issues raised by Simon et al (18, 19) for the statistical design of gene expression experiments, we have increased the size of the new prospective case-control studies. Third, the patients in our retrospective case-control studies were selected from two different sets of clinical trials coordinated by POG; even though these trials were related, it would be ideal to test more uniformly treated patients in future cohorts. Thus, we will refine our first expression classifier by profiling a prospective statistically-designed set of 150 uniformly treated pediatric ALL patients.

Development of Automated Quantitative RT-PCR Assays for Top Predictive Genes and Multivariate Statistical Analysis of the Power of Predictive Genes. The gene expression classifiers and top predictive genes identified from our prior retrospective and new prospective case control studies are compared. Automated quantitative RT-PCR assays are developed to measure the expression of these genes in a second independent validation cohort of 544 pediatric ALL patients selected from recently completed COG/CCG trials (1952/1961/1991). This new cohort has been statistically designed and powered by Dr. Sather to determine the predictive power and clinical utility of new gene classifiers. Working with Core B and Dr. Sather, we use univariate and multivariate statistical methods to determine if these genes provide additional independent prognostic information relative to the parameters currently in use for risk classification in pediatric ALL (age, race, WBC, cytogenetics, extramedullary disease, morphologic assessment of early marrow response, and quantitative levels of minimal residual disease at the end of induction therapy). If these genes and gene expression classifiers are confirmed to provide additional prognostic information, then new risk classification schemes will be developed and tested in a new classification study and associated clinical treatment trials that will be under development in COG.

Determine Whether the Genes Identified in the Pediatric ALL Classifer are Predictive of Outcome in Adult ALL (Jerald Radich, FHCRC).

Test Predictive Genes in A Prospective Adult ALL Cohort. Automated quantitative RT-PCR assays are used to test the predictive power of the 26 gene classifier in pre-treatment samples from a cohort of older adolescent and adult ALL patients being accrued to SWOG and COG trials, including COG/SWOG AALL0232.

Develop a Gene Expression Classifier and Identify Genes Predictive of Outcome in Adult ALL. To identify other predictive genes that may give insights for new therapeutic approaches in adult ALL, we use Affymetrix HG_U133 Plus2.0 Arrays to obtain gene expression profiles in a selected cohort 125 adult Ph-ALL patients registered to SWOG 9400. In this study, cases (failure within 4 years) are balanced with controls (CCR at 4 years) and other important prognostic factors (age, WBC). Cases are randomly assigned to training and test sets and genes predictive of outcome will be identified using statistical methods and multiple supervised machine learning algorithms with full cross validation in collaboration with Core B.

Relevant portion of the below cited references are incorporated herein. In addition, previously published WO 2004/053074 (Jun. 24, 2004) is incorporated by reference in its entirety herein.

### REFERENCES CITED

1. Pui C H, Relling M V, Downing J R. N Engl J Med 2004; 350:1535-1548.
2. Silverman L B et al. Blood 2001; 97:1211-1218.
3. Maloney K W et al. Leukemia 2000; 14:2276-2285.
4. Gaynon P S et al. Leukemia 2000; 14:2223-2233.
5. Nachman J B et al. N Engl J Med 1998; 338:1663-71.
6. Campana D et al. Acta Haematol. 2004; 112:8-15.
7. Borowitz M J et al. Leukemia 2003; 17(8):1566-72.
8. Neale G A et all. Leukemia 2004; 18:934-938.
9. Willman C L, Helman P, Veroff R L, Mosquera-Caro M P, Shuster J J, Kang H., Potter J W, Harvey R C, Martin S B, Davidson G S, Atlas S R, Andries E, Murphy M., Wang X, Ar K, Xu Y, Chen I-M, Wilson C S., Schultz F, Bedrick E J, Carroll A J, and Camitta B. Gene expression profiling reveals novel genes for risk classification and outcome prediction in pediatric acute lymphoblastic leukemia. N Engl J Med; 2005 (this manuscript reports the major findings in presented in Project 1; the manuscript was submitted in October 2004 and a significantly revised version was re-submitted; it will be provided to the LLS review committee if the accepted).
10. Additional array-based and computational methods published by our group can be found in references—, pg.
11. Helman P, Veroff R, Atlas, S R., and Willman C L. J Comput Biol; 2004: 11(4):581-615.
12. Guyon J I et al. Machine Learning 2002; 46:389-422.
13. Hjorth J S. Computer Intensive Statistical Methods, Validation, Model Selection, and Bootstrap. Chapman, Hall, London. 1994.
14. Ben-Dor A et al. J Comput Biol 2000; 7:559-584.
15. Tusher V G et al. Proc Natl Acad Sci USA 2001; 98:5116-5121.
16. Yeoh E J et al. Cancer Cell 2002; 1: 133-143.
17. Muramatsu H et al. J Cell Science 2004; 117(22) :5405-5415.
18. Simon R I, Radmacher M D, Dobbin K, McShane L. J Natl Cancer Inst 2003; 95:14-18.
19. Simon R I, Radmacher M D, and Dobbin K. Genetic Epidemiology 2002; 23:21-26.

1. A method for predicting therapeutic outcome in a leukemia patient comprising:
(a) obtaining a biological sample from a patient;
(b) determining in said sample the expression level for at least three gene products selected from the group consisting of midkine (neurite growth-promoting factor 2), CHST 10 (carbohydrate sulfotransferase 1 or HNK1-sulfotransferase), PHYN (phytanoyl-CoA hydroxylase), IFI44L (Interferon-induced protein 44-like, C1orf29), OPAL 1, CDK8 (cyclin-dependent kinase 8), DOK1 (docking protein 1-62 kD and downstream of tyrosine kinase 1) and ATP2C1 (ATPase-Ca++ transporting type 2C member 1) to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products; wherein an observed expression levels that is higher or lower than the control gene expression levels is indicative of predicted remission or therapeutic failure.

2. The method of claim 1 wherein said at least three gene products includes OPAL-1.

3. The method of claim 1 comprising determining the expression level for at least five of said gene products to yield observed gene expression levels; and comparing the observed gene expression levels for the gene products to control gene expression levels for the gene products selected from the group consisting of: (i) the gene expression levels for the gene expression products observed in a control sample; and (ii) predetermined gene expression levels gene products; wherein observed expression levels that are different from the control gene expression levels is further indicative of predicted remission.

4. The method according to claim 1 wherein an observed expression level which is greater than said control expression level is indicative of a favorable therapeutic outcome.

5. The method according to claim 1 wherein an observed expression level which is less than said control expression level is indicative of a favorable therapeutic outcome.

6. The method according to claim 1 wherein the expression levels of all eight genes are determined and wherein a favorable therapeutic outcome is predicted when the expression levels of midkine, CHST 10, PHYN, IF144L, OPAL 1 and CDK8 are above a control expression level and the expression levels of DOK1 and ATP2C1 are below a control expression level.

7. The method according to claim 5 wherein the expression levels of all eight genes determined and the expression level of at least one additional gene is determined, said additional gene being selected from the group consisting of ARHGEF (rho guanine nucleotide exchange factor 4); FYB (FYN-binding protein; FYB); DREBIN 1; SMAD 1; HABP4 (hyaluronan-binding protein);
Human cDNA FLJ30991 fis, clone HLUNG1000041; ITPR3 (inositol 1,4,5-triphosphate receptor, type 3); NMT2 (N-myristoyltransferase 2); C10orf26 (hypothetical protein FLJ20154); NPY (neuropeptide Y); JAG1 (jagged 1; Alagille syndrome); MONDOA (Mlx Interactor); EFN-B2 (ephrin-B2); LGALS1 (lectin, galactoside-binding, soluble, galectin 1); GST74 1 (glutathione-S-transferase omega 1); CCL5 (chemokine ligand 5)/RANTES; PRG1 (proteoglycan 1); CD44; and optionally, SPARC (secreted protein, acidic, cysteine-rich; osteonectin) and said expression level of said additional gene is compared to a control expression level.

8. The method according to claim 7 wherein the expression level of all of the additional genes is determined.

9. The method according to claim 7 wherein the expression level of ARHGEF, FYB (FYN-binding protein; FYB), DREBIN 1, SMAD , HABP4, Human cDNA FLJ30991 fis, ITPR3, NMT2, C10orf26, NPY, JAG1, MONDOA or EFN-B2 which is above a control expression level is indicative of a favorable therapeutic outcome and the expression level of LGALS1, GSTθ1, CCL5, PRG1 or CD44 which is below a control expression level is indicative of a favorable therapeutic outcome.

10. A method for predicting therapeutic outcome in a leukemia patient comprising:
(a) obtaining a biological sample from a patient;
(b) determining in said sample the expression level of gene products for the following genes: MIDKINE (neurite growth-promoting factor 2); ARHGEF (rho guanine nucleotide exchange factor 4); FYB (FYN-binding protein; FYB); CHST10 (carbohydrate sulfotransferase 10); DREBIN 1; SMAD1; HABP4 (hyaluronan-binding protein); PHYN (phytanoyl-CoA hydroxylase; Refsum disease); IFI44L (interferon-induced protein 44-like) (C1orf29); Human cDNA FLJ30991 fis, clone HLUNG1000041; ITPR3 (inositol 1,4,5-triphosphate receptor, type 3); NMT2 (N-myristoyltransferase 2)′ OPAL1; C10orf26 (hypothetical protein FLJ20154); NPY (neuropeptide Y); JAG1 (jagged 1; Alagille syndrome); MONDOA (Mlx Interactor); EFN-B2 (ephrin-B2); CDK8 (cyclin-dependent kinase 8); LGALS1 (lectin, galactoside-binding, soluble, galectin 1); DOK1 (docking protein 1, 62 kD, downstream of tyrosine kinase 1); GSTθ1 (glutathione-S-transferase omega 1); CCL5 (chemokine ligand 5)/RANTES; PRG1 (proteoglycan 1); CD44; ATP2C1 (ATPase, Ca++ transporting, type 2C, member 1); and optionally, SPARC (secreted protein, acidic, cysteine-rich; osteonectin) to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products;
wherein an observed expression levels that is higher or lower than the control gene expression levels is indicative of predicted remission.

11. The method according to claim 10 wherein the expression level of MIDKINE, ARHGEF, FYB, CHST10, DREBIN 1, SMAD1, HABP4, PHYN, IFI44L, Human cDNA FLJ30991 fis, ITPR3, NMT2, OPAL1, C10orf26, NPY, JAG1, MONDOA, EFN-B2 and CDK8, which is above a control expression level is indicative of a favorable therapeutic outcome and the expression level of LGALS1, DOK1, GSTθ1, CCL5, PRG1, CD44, ATP2C1 and optionally, SPARC which is below a control expression level is indicative of a favorable therapeutic outcome.

12. A method for screening compounds useful for treating acute lymphoblastic leukemia comprising:
(a) determining the expression level for at least three gene products selected from the group consisting of midkine (neurite growth-promoting factor 2), CHST 10 (carbohydrate sulfotransferase 1 or HNK1-sulfotransferase), PHYN (phytanoyl-CoA hydroxylase), IF144L (Interferon-induced protein 44-like, C1orf29), OPAL 1, CDK8 (cyclin-dependent kinase 8), DOK1 (docking protein 1-62 kD and downstream of tyrosine kinase 1) and ATP2C1 (ATPase-Ca++ transporting type 2C member 1) in a cell culture to yield observed gene expression levels prior to contact with a candidate compound;
(b) contacting the cell culture with a candidate compound;
(c) determining the expression level for the gene products in the cell culture to yield observed gene expression levels after contact with the candidate compound; and
(d) comparing the observed gene expression levels before and after contact with the candidate compound wherein a change in the gene expression levels after contact with the compound is indicative of therapeutic utility for said compound.

13. The method of claim 12 wherein said at least three gene products includes OPAL-1.

14. (canceled)

15. (canceled)

16. A method for screening compounds useful for treating acute lymphoblastic leukemia comprising:
(a) contacting an experimental cell culture with a candidate compound;
(b) determining the expression level for at least three gene products selected from the group consisting of midkine (neurite growth-promoting factor 2), OPAL1, HNK-1 sulfotransferase, phytanoyl-CoA hydroxylase (Refsum disease), cyclin-dependent kinase 8, docking protein 1 (62 kD and downstream of tyrosine kinase 1), ATPase (Ca++ transporting type 2C member 1), and optionally SPARC (secreted protein, acidic, cysteine-rich, osteonectin) in the cell culture to yield experimental gene expression levels; and
(b) comparing the experimental gene expression levels to the expression level of the gene products in a control cell culture, wherein a relative difference in the gene expression levels between the experimental and control cultures is indicative of therapeutic utility.

17. (canceled)

18. (canceled)

19. (canceled)

20. (canceled)

21. (canceled)

22. A method for predicting therapeutic outcome in a leukemia patient comprising:
(a) obtaining a biological sample from a patient;
(b) determining in said sample the expression level for at least three gene products selected from the group consisting of LGALS1/galectin, DOK1, GST1, CCL5/RANTES, PRG1, CD44, ATP2C1, SPARC to yield observed gene expression levels; and
(c) comparing the observed gene expression levels for the gene products to a control gene expression level selected from the group consisting of:
(i) the gene expression level for the gene products observed in a control sample; and
(ii) a predetermined gene expression level for the gene products; wherein an observed expression levels that is higher or lower than the control gene expression levels is indicative of predicted therapeutic failure.

23. The method of claim 22 wherein said at least three gene products includes SPARC.

24. The method of claim 22 comprising determining the expression level for at least five of said gene products to yield observed gene expression levels; and comparing the observed gene expression levels for the gene products to control gene expression levels for the gene products selected from the group consisting of: (i) the gene expression levels for the gene expression products observed in a control sample; and (ii) predetermined gene expression levels gene products; wherein observed expression levels that are different from the control gene expression levels is further indicative of predicted therapeutic failure.

25. The method according to claim 22 wherein an observed expression level which is greater than said control expression level is indicative of an unfavorable predicted therapeutic outcome.

26. The method according to claim 25 wherein a more aggressive traditional therapy or an experimental therapy is recommended for said leukemia patient.

27. (canceled)

28. (canceled)

29. (canceled)

30. (canceled)

31. (canceled)

32. (canceled)

33. (canceled)

34. The method according to claim 1 wherein said leukemia is B precursor acute lymphoblastic leukemia.
